(19) United States
(12) Patent Application Publication
(10) Pub. No.: US 2025/0292295 A1
(43) Pub. Date: Sep. 18, 2025

(54) SYSTEM AND METHOD FOR THERAPEUTIC EVENT MANAGEMENT AND VALUE ATTRIBUTION (PROOF OF VALUE) IN DECENTRALIZED HEALTH INFORMATION NETWORK

(71) Applicant: Lifeguard Health Networks, Inc., Radnor, PA (US)

(72) Inventors: Martin Carty, Wayne, PA (US); Stephen Malaby, Devon, PA (US)

(21) Appl. No.: 19/068,918

(22) Filed: Mar. 3, 2025

Related U.S. Application Data
(60) Provisional application No. 63/565,659, filed on Mar. 15, 2024.
(60) Provisional application No. 63/565,662, filed on Mar. 15, 2024.

(51) Int. Cl. G06Q 30/0283; G06Q 20/06; G16H 10/60; G16H 40/20; G16H 80/00

(52) U.S. Cl. CPC G06Q 30/0283; G06Q 20/06; G16H 10/60; G16H 40/20; G16H 80/00

(57) ABSTRACT

A distributed network system in a healthcare environment provides for proof of value transactions among various stakeholders like patients, providers, and payers. The system operates based on predefined intervention, valuation, and communication rules governed by a consensus contract. These rules determine interventions in response to therapeutic events, attribute valuations to interventions for different stakeholders, and regulate communications within the network. The system records therapeutic events, selects interventions, communicates them to relevant nodes, assessing valuations, and settling transactions according to the consensus contract. Different scenarios are outlined, such as interventions based on diagnoses or observations, including actions directed towards patients or providers, and rewards for achieving therapeutic goals. Overall, the system enables transparent and accountable transactions in healthcare by leveraging distributed ledger technology within a consensus-driven network.

### CROSS-REFERENCE TO RELATED APPLICATIONS

This application claims the benefit of U.S. Provisional Appl. No. 63/565,659 filed Mar. 15, 2024 and U.S. Provisional Appl. No. 63/565,662 filed Mar. 15, 2024, both of which are incorporated herein by reference in their entireties. Additionally, this application is filed concurrently with U.S. application Ser. No. 19/068,861 entitled “System and Method for AI-Assisted Therapeutic Micro-Interventions in Care at Home” (Atty. Dkt. No. 266-0007US) and U.S. application Ser. No. 19/068,782 and entitled “System and Method for Protecting Patients' Therapeutic Treatment Context Used for Training Artificial Intelligence Systems” (Atty. Dkt. No. 266-0004US), which are also incorporated herein by reference in their entireties.

### TECHNICAL FIELD

This disclosure relates generally to therapeutic monitoring and treatment of medical patients primarily outside of a hospital setting.

### BACKGROUND OF THE DISCLOSURE

Research studies and anecdotal reports agree that at-home medical treatment and recovery may be safer, cheaper, and more effective than traditional hospital care, but only if the patients are closely monitored. The benefits of home care may be especially important for patients who are vulnerable to infections that are common in hospitals or to other complications of inpatient care. In addition, some studies indicate that well-monitored healthcare at home may be more effective at improving patient outcomes than traditional hospitalization. Furthermore, most experts agree that home healthcare can save money for both patients and payer organizations, including governments.

Healthcare outside of a hospital or other dedicated medical environment benefits from fulsome patient surveillance because surveillance allows healthcare professionals to more granularly evaluate patient progress as well as a patient's adherence and response to therapies. Not surprisingly, there is a medical surveillance marketplace offering labor, techniques, and technology to monitor patients' health and safety when those patients are away from medical facilities. The technology employed in marketable solutions may include hardware, such as sensors, cameras, and other devices, which can track a patient's vitals, wellness, movements, or other activities. There is also software at either or both of the system or client application level, which can help a patient report on subjective matters and help enforce the proper administration of therapies. In all, these technologies provide healthcare professionals with real-time or near-real-time information about a patient's condition and other medically relevant activities, which allows more accurate and timely medical interventions.

Certain solutions for remote patient monitoring are static, hard-coded data models that require universal updates across all parties and global adherence to a single definition. This monolithic approach limits best practice logic and/or customization that may be added by users (e.g., providers or other participants in the healthcare marketplace) with more relevant knowledge regarding the needs of patient cohorts or individual patients. By hindering the flexibility of downstream healthcare providers, researchers, and administrators, such static and hard-coded models lock all participants into the same data model universally. That result may hinder differentiation of remote surveillance services, may limit the availability of more customized patient-centric care, and may potentially burden the clinical teams with manual application of customizations.

Currently, healthcare services are shifting to value-based contracts between insurers, health plans, and managed care teams. These entities want to address the inherent challenges in coordinating care for at-risk Medicare populations outside of clinical settings. Accordingly, providers and payers seek to improve performance and meet quality and cost targets by getting ahead of clinical risks, engaging patients, and effectively optimizing healthcare costs and revenue cycle. Unfortunately, the present state of the art limits the information available to payers and providers for satisfying value-based contracts. In fact, good evidence of care occurring at a patient's home (tolerance, adherence, and risk) is not available to providers for them to satisfy value-based contracts. Typically, payers and providers only have access to the clinical observations captured episodically in the Electronic Medical Record (EMR).

What is needed are systems and methods to overcome the challenges of at home surveillance and patient/provider interaction between clinical encounters.

### BRIEF DESCRIPTION OF DRAWINGS

FIG. 1 illustrates an example of a health information network according to the present disclosure.

FIG. 2 illustrates a proof of value system utilized in a distributed health information network system.

FIG. 3 illustrates a flowchart of a proof of value process according to the present disclosure.

FIG. 4A illustrates an example of the distributed network system being used for therapeutic engagement.

FIG. 4B illustrates a portion of the proof of value process for the therapeutic engagement example in FIG. 4A.

FIG. 5A illustrates an example of the distributed network system being used for therapeutic tolerance and safety.

FIG. 5B illustrates a portion of the proof of value process for the therapeutic tolerance and safety example in FIG. 5A.

FIG. 6 illustrates an example of the distributed network system being used for therapeutic consumption.

FIG. 7A illustrates an example of the distributed network system being used for therapeutic reward.

FIG. 7B illustrates a portion of the proof of value process for the therapeutic reward example in FIG. 7A.

FIG. 8A illustrates an example of the distributed network system being used for AI-assisted valuation.

FIG. 8B illustrates a portion of the proof of value process for the AI-assisted valuation example in FIG. 8A.

FIG. 9 illustrates a processing platform for creating training data to be used in training an AI model in a computing environment.

FIG. 10 illustrates a process for training and deploying a deep neural network.

FIG. 11 schematically illustrates components of network nodes for the distributed network system.

### DETAILED DESCRIPTION OF THE DISCLOSURE

U.S. Publication No. US2014/0207486 to Carty et al. and assigned to Lifeguard Health Networks, Inc. is hereby incorporated by reference in its entirety as background information and to aid in the understanding of more advanced concepts discussed herein.

Systems and methods described herein disclose a network enabled (e.g., TCIP/IP enabled) continuity of care network, including one or more computing devices, sensors, and systems for communications and data sharing. These networks enable people on a caregiver team to receive and send medically relevant communications with a patient or a patient representative in response to health events, incidents, questions, observations, trends, and the like. A continuity of care network may be doctor-initiated and may be payer or insurer approved, and the continuity of care network is part of one possible network arrangement of this disclosure. In many arrangements, patients are invited onto the system by a healthcare provider or a medical-related entity with a relationship to the patient's care. Because healthcare often involves a support team around the patient, arrangements of the disclosed network system allow patients to invite family, friends, and other health advocates to join the system as a patient representative. One or more computing devices for each system participant may be joined into the network/system to run or access software related to the techniques herein and access associated functionality.

The disclosed system proposes technology and solutions through a collaborative care network, including devices having software made available for use by various participants in the healthcare ecosystem and marketplace. For example, depending upon the arrangement, software delivering all or part of the solution may reside on mobile devices, computers, thin clients, or intelligent devices of any kind that are commonly used by patients, patient representatives, healthcare professionals, insurers, government, medically related entities such as institutions, health systems, research entities, and all manner of healthcare provider organizations. As implemented, the software may have multiple widespread application areas. Healthcare providers and other healthcare related entities (such as insurers and institutes) may use a version of software (e.g., application) adapted for endpoint operating systems such as Microsoft Windows or Mac OS, or server operating systems (e.g., for SaaS solutions) such as Linux or Windows Server. In addition, some embodiments allow providers and other entities access to software functionality through (i) mobile applications (e.g., IOS, Android, Java); (ii) a web portal accessed through a web browser on any device; or (iii) any information access and/or delivery system such as virtual or augmented reality tools. In some embodiments, these options may be facilitated through known client-server protocols using computer servers in a data center, public cloud, or other known computing resources.

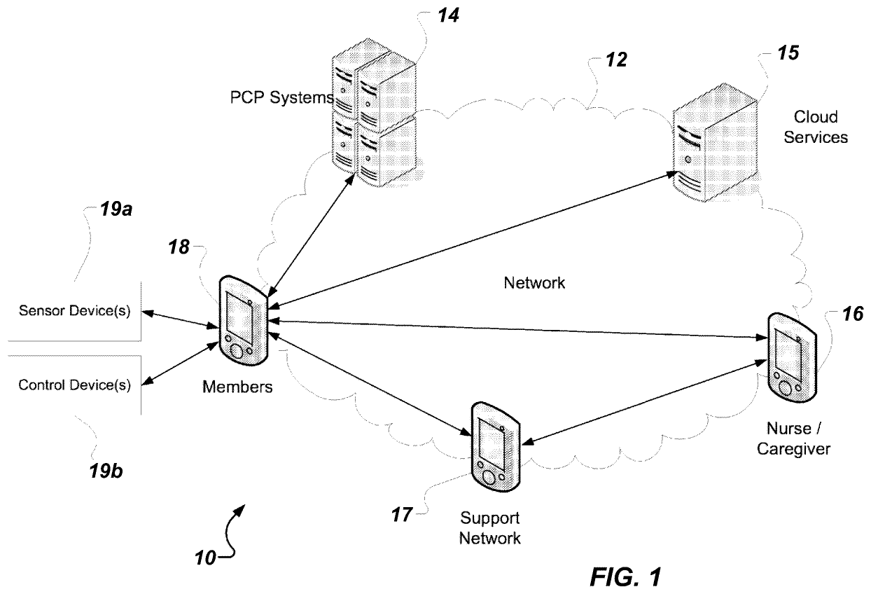

FIG. 1 shows an exemplary continuity of care network system 10. As shown in FIG. 1, the network system 10 is decentralized with computing power, data capture and monitoring and other functionality distributed and available at each of the various components. Patients or their representatives typically interface with/connect to the network system 10 with patient devices 18. Patient device 18 may be an edge-of-network computing device (e.g., a computer or mobile device such as a smartphone or tablet), optionally configured to monitor patient data based upon a specified health service policy. For example, a device may monitor the patient's biotelemetry, location, or other items depending upon the capability of the device. The patient's device (e.g., client device) 18 may also receive inputs from other devices such as sensors 19a or control devices 19b. Patient device 18 may transmit patient data across one or more networks 12 to other devices and systems in the network system 10, such as one or more representative devices (e.g., client device) 17, a nurse or other caregiver device (e.g., client device) 16, one or more primary care provider systems 14, and servers/databases such as cloud services systems 15. Of course, network system 10 may include any number of additional devices and systems or may omit one or more devices or systems shown in FIG. 1.

Patient representative devices (e.g., client device) 17 may be used by those appointed by or on behalf of the patient, such as trusted family, friends, and other caregivers of a patient to assist with daily therapy compliance, program adherence, communications, health management, and the like. Network system 10 may include multiple patient representative devices 17 coupled to (i.e., in communication with) a patient device 18, thereby providing a few-to-one or many-to-one support network for the patient. By including multiple people's devices with customized privacy and intervention alert levels within the network, the responsibility of monitoring patient data may be distributed amongst a patient's family or other care representatives or network participants. Additionally, such a group may provide monitoring redundancy, for example if one representative does not have access (e.g., internet access) others in the patient's support network may monitor patient's data. In addition, aspects of the network system 10 associated with a given patient may be dynamic, expanding and contracting in response to a patient's needs, schedule, and the like.

Representative devices 17 and nurse/caregiver device 16 may be any type of computing devices, such as any computing endpoint or mobile computing device, thereby enabling monitoring the patient substantially all times. Devices on the network system 10 may receive substantially real-time health data from patient device 18 to allow for timely and effective intervention mechanisms or other actions.

Network system 10 may also include one or more healthcare provider systems 14 (e.g., PCP—or primary care provider systems). These provider systems 14 may periodically receive patient data from patient device 18, optionally as relayed through one or more servers. Medical practitioners may use provider systems 14 to diagnose patients, monitor effectiveness of therapy regimens, access EHR systems and facilitate similar functions or other tasks related to the provision of healthcare. Network system 10 may allow medical providers to monitor and diagnose many patients while simultaneously allowing designated family, friends, nurses, and the like to also monitor the patient's therapy regimen compliance, potential emergency and other situations or statuses.

Network system 10 may include or access cloud resources 15. Cloud resources (e.g., cloud service systems) 15 may provide a broad range of services, including using autonomous agents (for example, bots) for monitoring patient data received from patient device 18 and, at times, responding to patient data by sending a message or control signal to patient device 18 or contacting escalation services (e.g., “911”) according to rules defined by the applicable health service policy. Autonomous agents may provide continuous (i.e., 24/7) monitoring and may trigger appropriate responses in response to any type of event (these responses could include providing a message to a patient in response to a missed dose, contacting patients in response to a trending health incident, or contacting emergency services in the event of a detected emergency, for example). Cloud resources 15 may also include databases, dynamic resource registries, health service policies, monitoring services, data analytics, on-boarding and patient activation services, and the like.

A patient device 18 may also contain health service policy logic and algorithms configured to monitor continuously a patient's health events. In certain embodiments, local health service policy logic may trigger appropriate responses to any type of event.

Network system 10 may also illustrate a functional and communication relationship between many computing devices. However, each computing device's configurations may be governed by a patient-centered policy templates discussed below. In many embodiments, the patient-centric policy template provides a functional hierarchy and structure to the relationship of the computing devices making up network system 10, including providing escalation and intervention services and payer approval/preapproval services.

The devices and systems comprising network system 10 may be operatively coupled by one or more networks 12. Network 12 may include one or more of the Internet, local area networks (“LANs”), wide area networks (“WANs”), mobile telecommunications networks (e.g., 3G and 4G networks), and any suitable type of communication mediums. As illustrated in FIG. 1, the network system 10 may include one or more decentralized computing/communication devices or systems (14, 15, 16, 17, 18) configured to communicate with one or more other computing devices. For example, each of a plurality of computing devices may use mobile health client software executed on a computing device using a microprocessor, random access and fixed memory, input, and output devices, and facilitating common communication protocols such as Bluetooth, Wi-Fi and 4G and/or 5G data and voice access. Some configurations may capture and store patient data in a distributed fashion, for example, on the patient's computing device, rather than storing and accessing data on a central server or plurality of distributed servers.

Each communication device (16, 17, 18) in the decentralized network 12 may originate communications to any other communication device in the decentralized network. Thus, the decentralized network may allow for scaling of compliance and intervention tasks. For example, upon detection of a compliance event, the patient device 18 or a connected device/server may determine if the detected compliance event should be escalated to an incident such that a notification or alert should be sent to a health professional or patient representative. Thus, configurations may allow for a direct one-to-many communication notifying any of the system participants of a compliance event or other event or status. The embodiments also allow for many-to-one communications, for example, from health care providers or patient representatives to the patient. Certain configurations may also provide for direct communication between the patient communication device 18 and intervention services if the there is a determination that that level of escalation is desired, for example a healthcare professional or even 911.

In addition to the patient side solutions for the patient and his/her representatives, some configurations of the disclosure may employ a decentralized patient network operations console (not shown in FIG. 1) for healthcare providers as well as for the non-professional responsible for managing multiple patients. A console/dashboard may be dynamic and driven by functional role, permissions and availability of resource enabling real-time monitoring, intervention, and mobilization of resources in response to any health event/status/escalation. This type of console functionality may enable a healthcare professional (physician, nurse, nurse practitioner, nutritionist, an allied health professional, and/or a health coach) to have a personalized dashboard specific to their role and expertise so they may provide continuity of care services concurrently to a number of members each with their own discrete, individually tailored physician directed parameters of therapeutic care.

Configurations contemplate the use of sensors that determine vitals, wellness, or other health-relevant information about a patient. Examples of these types of sensors are thermometers, scales, blood oxygen sensors, blood pressure cuffs, and similar devices that provide telemetry/biotelemetry readings or any therapeutically relevant status or reading related to a patient. The patient's computing device may operate as a sensor or may receive data from the sensors in real-time, near real-time, periodically, or event-based, when a sensor reading or measured trend passes a threshold, etc. Certain configurations may use standard (e.g., cross-platform (e.g., iOS, Android, BlackBerry OS, Windows Phone 7, etc.)) plug-ins to interact with devices. Additionally, configurations may be deployed using existing or to-be-developed computing systems and communication platforms.

Medical-related organizations may be onboarded to configurations of the system either by invitation from the system administrator or by enrolling in a program associated with the system. Patients may be onboarded in any manner for onboarding individuals to software resources. For example, they may be onboarded to the network system 10 and become network participants using a link or invitation provided, for example, by a healthcare provider that already is a participant. A patient representative may be similarly onboarded or alternatively receive a link or invite from the patient and facilitated by the patient's mobile application. Patient representatives may be designated with one or more particular roles, thereby defining their capabilities in the system.

Configurations of the disclosure contemplate the handling of patient data consistent with government regulations (e.g., HIPPA) and patient privacy expectations. In most configurations, all patient health information (PHI) is protected by both access restrictions and encryption. For certain configurations, the access limitations are determined by the patient's permissions and/or by an associated healthcare provider. Encryption or other data protection techniques are employed relative to best practices and expected to evolve over time.

In some configurations, all data in the system is maintained in one or more logical location, such as a database, designated or agreed by the system administrator. Alternatively, more sensitive data may be maintained closer to and in storage controlled by the data owner (e.g., healthcare provider or patient), with less sensitive data flowing to a centralized logical database or server.

Configurations of the disclosure provide for access to data being designated based upon one or more of a per datum basis, a per patient basis, a per provider basis, a per publisher basis, a per subscriber basis or otherwise. In one or more configurations, multiple entities may have access to the patient data or portions thereof. For example, for a certain patient policy template, the patient data may be shared with multiple providers engaged in the treatment of the patient; perhaps a primary care physician, a medical oncologist, a surgeon, and a physical therapist. In some configurations, entities that are not providers may also have access to the data or portions thereof, such as family members, caregivers, insurers, researchers, system developers, health networks, institutions, or organizations.

To improve performance and meet quality and cost targets, providers and payers leverage technology disclosed herein, including distributed ledgers and remote patient care systems, to get ahead of clinical risks, to engage patients, and to effectively optimize healthcare costs and revenue cycle. Therapeutic evidence of activities of a patient's care at home (tolerance, adherence, and risk) is made available to payers and providers for them to satisfy value-based contracts. To do this, a proof of value system as disclosed herein is used in the health information network.

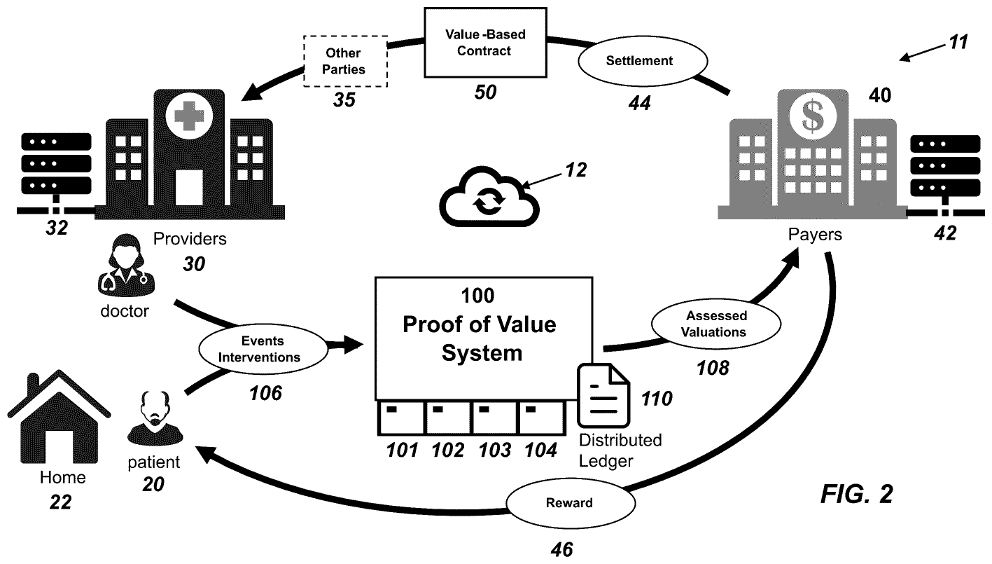

FIG. 2 illustrates a proof of value (PoV) system 100 utilized in a health information network system 10. The PoV system 100 works with network nodes 11 over one or more networks 12. The network nodes 11 are associated with members in the healthcare environment, including, for example, payers 40 having payer systems or “node” 42, health providers 30 having provider systems or “node” 32, and patients 20 having patient systems or “node” 22.

Additionally, other parties 35 may be included in the network of nodes 11 as necessary to deliver care. In one example, a Beneficiary and Family Center Care (BFCC) organization may be one such party 35 that delivers care management services to patients and families and that coordinates the delivery of community resources to aid the patients' care. Accordingly, the PoV system 100 is configured to be inclusive of all care-related interactions between patients and professionals.

The PoV system 100 uses the decentralized health information network system 10 that spans distributed data sources and systems 22, 32, 42, even extending into patients' homes. In that sense, the PoV system 100 can be a cloud-based system or can be a decentralized or distributed system that utilizes and/or is spread across the distributed data sources and systems 22, 32, 42. As used herein, the PoV system 100 may be referenced as a distributed network system, a distributed system, or simply the “disclosed system.”

The distributed network system 100 coordinates a proof-of-value consensus governed by a value-based consensus contract 50 between payers 40 and providers 30 for patients 20). Briefly stated, the PoV system 100 receives interventions or actions from patients 20 and providers 30, determines the assessed value of those interventions, and provides the assessed value to payers 40. Based on the assessed value, the payers 40 can provide an appropriate settlement 44 according to the value-based consensus contract 50 to the providers 30. Additionally, the payers 40 can provide an appropriate reward to the patients 20 based on the assessed value.

In the present disclosure, references are made to an “intervention” or an “action.” These can be used interchangeably. In customary understanding, an intervention may refer to a medical-related, therapeutic-related, or other type of action, but that is not strictly the case according to the context of the present disclosure. As disclosed herein, for instance, an “intervention” or an “action” in response to a patient's diagnosis can include any of the customary, medical-related actions. Additionally, an “intervention” or an “action” in response to a patient's diagnosis can include additional practices, such as, for example, assigning the patient to a care plan for that condition and can further include assigning a follow up call in a designated time period (e.g., one week) to be made from the care manager to assist the patient. Additionally, any plans for interventions and actions according to the present disclosure may be multi-fold and may be conducted in parallel.

In general, the disclosed system 100 includes a therapeutic module 101, a correlation module 102, a rules engine or rules 103, and a distributed ledger or blockchain module 104, which uses a Proof of Value consensus protocol to capture direct (unrepudiated) evidence that patient interactions (interventions or actions 106) have been completed. In turn, the Proof of Value consensus protocol of the disclosed system 100 attributes predefined valuations 108 for each evidenced intervention or action 106. Ultimately, the disclosed system 100 can be used to substantiate the value of the predefined valuations 108 for each evidenced intervention or action 106 and can be used to corroborate and interrogate digital evidence of therapeutic intervention or action 106.

Each intervention or action 106 of a medical-related, therapeutic-related, or other type of event for the PoV consensus protocol is uniquely identified in a distributed ledger 110 of the disclosed system 100. The distributed ledger 110 includes information about an origin (i.e., physician/diagnosis or patient/therapeutic escalation), a mediator (i.e., patient/care plan acceptance or physician/prescription), and validators (i.e., health plan administrator, healthcare provider, and health consumer/patient). The distributed ledger 110 also attributes measured valuations surrounding the performance, the health quality, and the outcomes for the therapeutic events (e.g., medical-related events, educating, literacy, caregiving, etc.) and interventions or actions 106. The valuations 108 are governed by the consensus contract 50 between the payers 40 and providers 30. In addition to providing the distributed ledger 110 for the assessed valuations 108 of the event interventions, the disclosed system 100 acts as a therapeutic event aggregator, which has a number of uses on its own.

At a minimum, the distributed ledger 110 is preferably protected, having restricted read/write access privileges to the information contained therein. Additionally, the distributed ledger 110 is also preferably self-proving, indicating the source of information, the history of the information, and the changes to the information. More particularly, the distributed ledger 110 is a blockchain using a decentralized, distributed ledger technology. In that sense, the distributed ledger 110 can have a continuously growing list of records, called blocks, which are linked together and secured cryptographically.

The distributed ledger 110 is decentralized so that the devices, systems, and other “nodes” 11 in the decentralized healthcare network system 10 can operate on the distributed ledger 110. Each node 11 has a copy of the entire blockchain, and no single entity has full control over it. This decentralized nature enhances transparency, security, and resilience.

For a given patient, a new distributed ledger 110 can be constructed for each medical-related event, diagnosis, or other discrete element and may contain a record of all transactions that have ever occurred on the network related to that discrete element. Alternatively, for a given patient, the distributed ledger 110 can be persistent and may contain a record of all transactions that have ever occurred on the network. Either way, these transactions are grouped into blocks, and each block is linked to the previous one, forming a chain.

Every node 11 on the network system 10 has a copy of this distributed ledger 110, ensuring that the information is distributed and replicated across the network system 10. Due to the existence of medical information contained, the distributed ledger can preferably use a permissioned blockchain technology, which restricts access to authorized participants. Permissionless blockchain technology may be used in some circumstances.

The data recorded in a block and added to the distributed ledger 110 is secured from alteration or tampering by using cryptographic hashing. In this technique, each block contains a unique cryptographic hash of the previous block. Any attempt to alter a given block requires changing all subsequent blocks, which is computationally infeasible and would require consensus from the majority of nodes 11 in the network system 10.

As disclosed herein, the decentralized health information network system 10 disclosed herein employs a consensus mechanism (i.e., Proof of Value (PoV)) to validate and agree on the state of the distributed ledger 110. The consensus mechanism determines how new blocks are added to the distributed ledger 110 and defines how conflicts are resolved. These mechanisms ensure that all nodes 11 in the network system 10 reach agreement on the validity of transactions without the need for a central authority to resolve conflicts.

As disclosed herein, the decentralized health information network system 10 disclosed herein supports self-executing “smart” contracts—i.e., the consensus contract(s) 50 described herein. Accordingly, the contract terms are directly written into code used in the network system 10 so the code can automatically enforce and execute the contract terms of the agreement once predefined conditions have been met. By having these feature, the disclosed system 100 can use digital decentralized applications (DApps), which can run on the network system 10 of nodes 11.

The PoV consensus protocol of the disclosed system 100 is completed once “proof of stake/action” is registered in the network system 10—i.e., by a healthcare provider 30 (e.g., a coded encounter, a referral, and/or a prescription) and/or by a health consumer (e.g., a patient 20 or a patient's authorized proxy who may schedule appointments and/or may take certain therapeutic actions). In the end, to achieve Proof of Value consensus, a given therapeutic event (e.g., medical-related event) intervention requires consensus among all three stakeholders (e.g., payer 40, provider 30, and patient 20 (or patient proxy).

Using the disclosed system 100, for example, a provider 30 of record can access the therapeutic module 101 and can prescribe a digital therapeutic for a diagnosed condition for a patient 20. The correlation module 102 of the disclosed system 100 performs a correlation to identify a therapeutic event (e.g., medical-related event), and the rules 103 of the rules engine integrates policy-based care management at the point of care, such as in the patient's home. The therapeutic module 101 and rules 103 enable early signal detection of patient health status and deterioration. The rules 103 of the engine preferably satisfies CMS performance and preferably makes quality measurements associated with the value-based consensus contract 50. To do this, the disclosed system 100 provides data related to health status and therapeutic adherence and tolerance between clinical encounters, and the disclosed system 100 quantifies the impact of earlier (lower cost) health interventions.

The ledger module 104 constructs a distributed ledger 110 having the condition identification, including the provider 30, patient 20, and the condition and having other information (e.g., intervention or action 106 and the assessed valuation 108) for the proof of value discussed herein.

This decentralized health information network system 10 can take feeds from provider RCM systems, pharmacy claims systems, payer risk stratification solutions, as well as the remote care management and monitoring solutions in the home. This decentralization enables unique digital pathways for micro-interventions (adjustments in care) as well as value attribution based upon the PoV consensus of the provider 30, patient 20, and payer 30 for a “therapeutic course of action”.

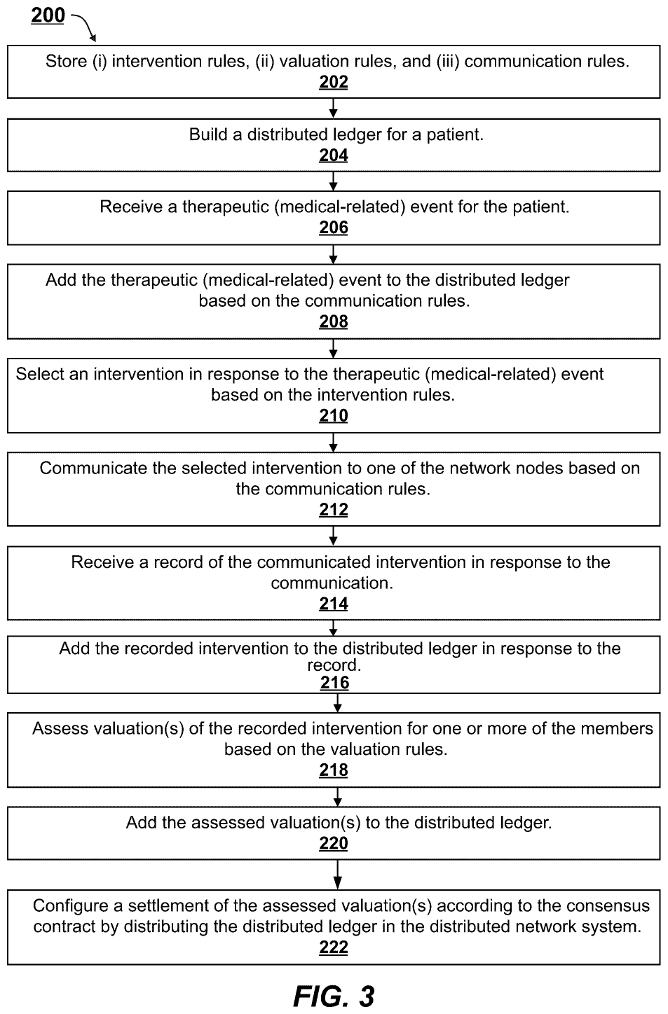

FIG. 3 illustrates a flowchart of a process 200 according to an example of the present disclosure to fulfill proof of value transactions according to a consensus contract. In this example, one or more process blocks of FIG. 3 may be performed by the PoV system (e.g., distributed network system) 100 on the network system 10 discussed above. For further understanding, reference to elements in FIGS. 1-2 may be used in the discussion of the disclosed process 200.

In the disclosed system 100 involving various network nodes 11, proof of value (PoV) transactions are fulfilled according to a consensus contract 50 using the disclosed process 200. The PoV transactions start with the therapeutic events (e.g., medical-related events) for patients 20 and encompass the interventions or actions 106, assess valuations 108, and settlements/rewards 44, 46.

As noted above, the network nodes 11 are at least associated with patients 20, providers 30, and payers 40. The disclosed system 100 operates in various healthcare environments involving various network nodes 11 and therapeutic events, ultimately ensuring seamless fulfillment of PoV transactions (106, 108, 44, 46) according to the established consensus contract 50.

Briefly, the providers 30 can include one or more of a healthcare provider, a hospital, a doctor, a medical practitioner, a medical supplier, a pharmacy, etc. The payers 40 can include one or more of a health management company, a medical insurer, an insurance company, a health plan administrator, etc. The medical-related events can include one or more of a medical diagnosis, a medical observation, a patient's biotelemetry data, etc. Meanwhile, the interventions can include one or more of a therapeutic protocol, a digital therapeutic program, a directed action, a communication from a care navigator or a social worker, a reward for achieving a therapeutic goal, an increase in care giving support in the home, providing medical equipment to improve monitoring, an adjustment to a patient care plan (i.e., the plan's scope, schedule, or thresholds for intervention), etc.

The process 200 involves several steps, all orchestrated by the disclosed system 100. Initially, the disclosed system 100 stores a multitude of rules 103—intervention rules, valuation rules, and communication rules—governed by a PoV consensus protocol from the consensus contract 50 (Block 202). As will be appreciated, the rules 103 are stored in one or more databases of the disclosed system 100. These rules 103 define specific interventions for defined therapeutic events (e.g., medical-related events), attribute valuations to the members (e.g., patients, providers, etc.) in the healthcare environment for these interventions, and outline communication protocols for interacting with each network node 11 for the interventions.

For example, the intervention or action rules are used to detect and select specific interventions or action in response to defined events, such as therapeutic events, medical-related events, or other types of events. The intervention or action rules can generally define how each event (e.g., a medical-related event, a therapeutic-related event, or other type of event) is detected and how any one or more specific interventions or actions are subsequently selected. As noted above, reference to “intervention” and “action” may be used interchangeably herein. Additionally, the “event” referenced herein need not be “medical-related” only, as other types of events may be involved. For instance, the hospitalization of a spouse giving care to the patient can constitute an “event” (e.g., a “therapeutic-related event”) according to the present disclosure. The intervention or action rules are used to detect and select specific intervention(s) or action(s) in response to such defined therapeutic-related events. The rules can generally define how each therapeutic-related event is detected and how any one or more specific actions are subsequently selected. For example, the therapeutic-related event involving the hospitalization of a spouse giving care to the patient may involve a plan of action(s) including scheduling an additional communication from a health plan care manager and assigning a part-time caregiver to assist the patient. The valuation rules can generally assess recorded interventions or actions by defining how the recorded interventions or actions are detected and how the recorded interventions or actions are subsequently valued. The intervention or action and valuation rules make up a form of business logic implemented at the disclosed system 100.

For their part, the communication rules generally govern how the disclosed system 100 interfaces with network nodes 11, such as defining how the disclosed system 100 sends and receives communications. In that sense, the communication rules are engagement and collection rules defining how the disclosed system 100 engages with network nodes 11 and how the disclosed system 100 collects information related to therapeutic events (e.g., medical-related events) and inventions from the network nodes 11 or from elsewhere.

During the process 200, a distributed ledger 110 for a given patient 20 is constructed using one or more processors within the disclosed system 100 (Block 204). Building the distributed ledger 110 starts with receiving an event (e.g., therapeutic event, a medical-related event, or other type of event) for the patient 20 (Block 206) and then adding or incorporating the event into the distributed ledger 110 according to the communication rules (Block 208). The event can be received from any number of sources, such as from any one of the network nodes 11. As a few examples, a medical-related event can come in the form of a diagnosis from a provider 30, an observation from the patient 20 or patient representative, or biotelemetry data from a device at the patient's home. As another example, a therapeutic-related event can come from the payer (40) detecting the hospitalization of a spouse that was providing caregiving to the patient.

An appropriate intervention or action 106 for the therapeutic-related, medical-related, or other type of event is selected based on the intervention rules (Block 210). The intervention or action 106 is then communicated to the relevant network node(s) 11 based on the communication rules (Block 212). The intervention or action 106 can be communicated to any appropriate one or more of the network nodes 11 depending on the intervention or action. For example, some forms of intervention or action 106, such as a digital therapeutic program, can be communicated directly to the patient 20, while other forms of intervention or action 106 may be communicated to other network nodes 11 or other destination.

When a record of the communicated intervention or action 106 is then received at the disclosed system 100 (Block 214), information of the intervention or action 106 is added or incorporated into the distributed ledger 110 (Block 216). The record can take many forms, including an acknowledgement, a consent, or other indication directly from the recipient member's network node 11, indirectly from another member's network node 11 related to the particular intervention, or from another source.

The process 200 then involves evaluating the intervention's impact on involved member(s) (e.g., patient, provider, etc.) by assessing one or more given valuations 108 of the recorded intervention or action 106 for a given one or more of the members (e.g., patient, provider, etc.) based on the valuation rules (Block 218). Information about these assessed valuations 108 is then added to the distributed ledger 110 (Block 220).

The disclosed system 100 further ensures that a settlement 44 (or reward 46) of these assessed valuations 108 is fulfilled for the members (e.g., patient, provider, etc.) in accordance with the consensus contract 50 by distributing the distributed ledger 110 across the network system 10 and configuring the settlement 44 (or reward 46) of the assessed valuations 108 accordingly (Block 222).

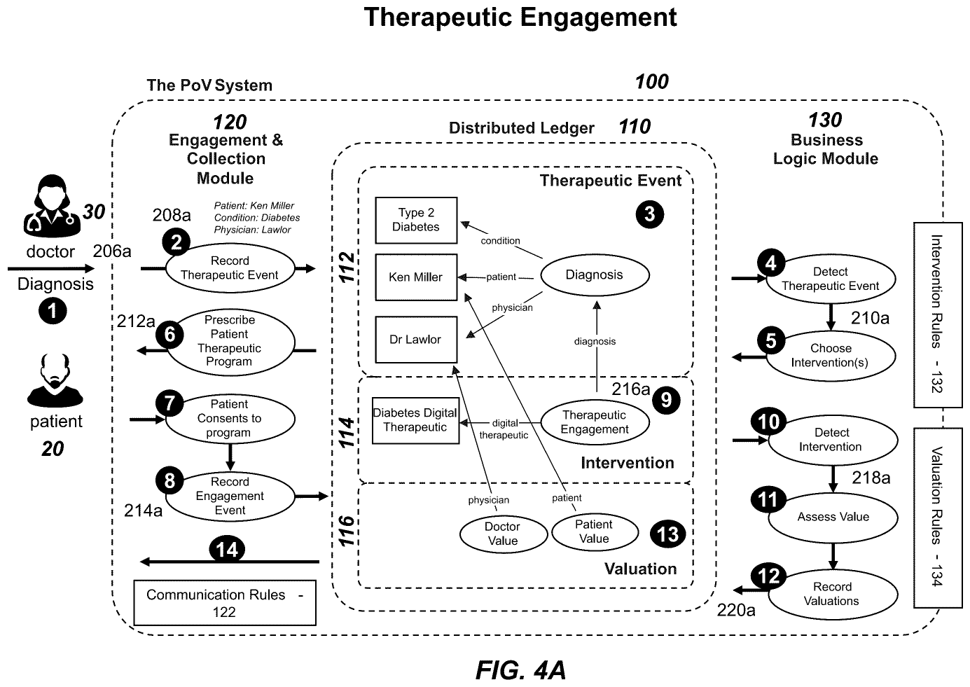

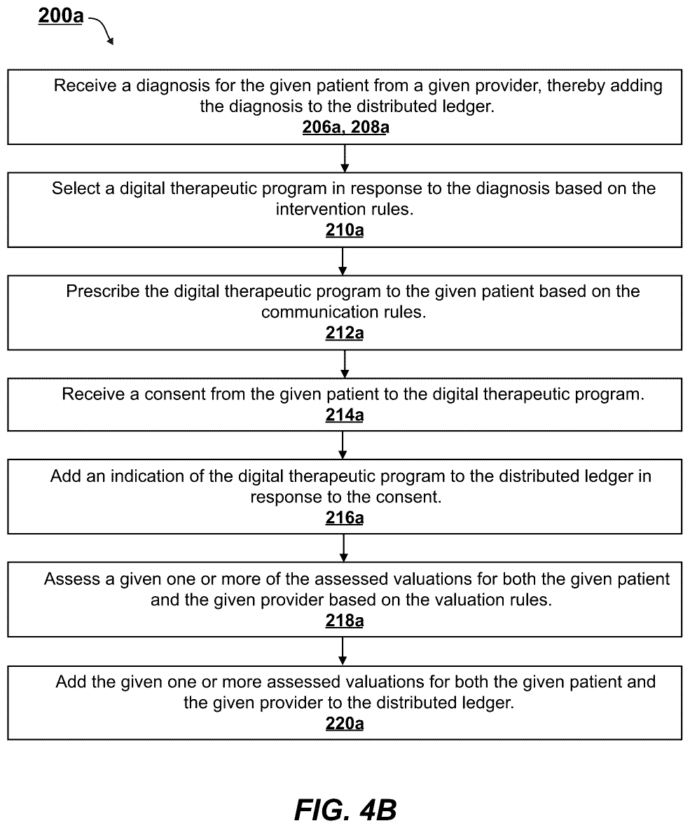

In specific instances, such as receiving diagnoses or observations for given patients 20, the disclosed system 100 selects interventions or actions 106, communicates them to the relevant network node(s) 11, records their completion, and assesses valuations 108 based on the intervention rules and the valuation rules outlined earlier. FIGS. 4A-4B, 5A-5B, 6, and 7A-7B described below show examples of several instances.

The process 200 may include additional implementations, such as any single implementation or any combination of implementations described below and/or in connection with one or more other processes described elsewhere herein.

Although FIG. 3 shows example blocks of the process 200, the process 200 in some implementations may include additional blocks, fewer blocks, different blocks, or differently arranged blocks than those depicted in FIG. 3. Additionally, or alternatively, two or more of the blocks of the process 200 may be performed in parallel.

FIG. 4A illustrates an example of the PoV system (e.g., distributed network system) 100 in more detail. Here, the disclosed system 100 is being used for therapeutic engagement of a patient 20. For further understanding, reference is also made to FIG. 4B, which illustrates a portion of the PoV process 200a for the therapeutic engagement example in FIG. 4A.

The disclosed system 100 uses an engagement and collection module 120 (having the disclosed communication rules 122) and uses a business logic module 130 (having the intervention rules 132 and valuation rules 134) to select intervention, to perform valuations, and to construct a distributed ledger 110, which is then used throughout the disclosed system 100 and network system 10. When constructed, the distributed ledger 110 for a given patient 20 includes information about an underlying therapeutic event (e.g., medical-related event) (section or block 112), an intervention selected to address that therapeutic event (section or block 114), and an assessed valuation for the intervention (section or block 116).

The engagement and collection module 120 uses the communication rules 122, which define how the disclosed system 100 interfaces with doctors 30 and patients 20, including how a therapeutic event is recorded, how a therapeutic program is prescribed to the patient 20, and how the patient 20 consents to the program. The business logic module 130 uses the intervention rules 132, which define how therapeutic events are detected and how interventions are in turn selected. Additionally, the business logic module 130 uses valuation rules 134, which define how an intervention is detected and how that intervention is then valued.

During therapeutic engagement, a provider 30 (e.g., doctor) makes a diagnosis of the given patient 20 (Step 1). Using the communication rules 122, the engagement and collection module 120 records the therapeutic event (Step 2) by adding it to the distributed ledger 110 (Step 3). In the provided example, the physician (Dr. Lawlor) has diagnosed the patient (Ken Miller) with a condition (Type 2 diabetes). The information of this therapeutic event is added to the therapeutic event section 112 of the distributed ledger 110.

The business logic module 130 of the disclosed system 100 now detects the therapeutic event (Step 4) and chooses intervention(s) for the detected medical-related event (Step 5). Using the intervention rules 132 governing therapeutics available for the therapeutic event (e.g., diagnosed condition), a set of interventions may be selected and initiated based on the type of therapeutic (medical-related) event. In this example, the disclosed system 100 initiates the assignment of the patient 20 to a digital therapeutic program for diabetes. Like all intervention logic, metrics provided to the system 100 are compared to thresholds in the interventions rules 132 to determine the resulting intervention to be selected. The disclosed system 100 then matches the candidate criteria to a specific therapeutic for the resulting intervention.

Before the selected intervention(s) are actually added to the distributed ledger 110, the engagement and collection module 120 interacts with the patient 20. In particular, the disclosed system 100 prescribes the digital therapeutic program, which is communicated to the patient 20 for acceptance (Step 6). The system 100 obtains the patient's acceptance (consent) to the therapeutic program (Step 7). In turn, the system 100 records the engagement event (Step 8) and records the intervention into the intervention section 114 of the distributed ledger 110 (Step 9).

In response to the therapeutic engagement by the patient 20, the business logic module 130 uses the valuation rules 134 to detect the intervention (Step 10), assess the valuation of the intervention (Step 11), and record the valuation(s) into the distributed ledger 110 (Step 12). The valuations are added to the valuation section 116 of the distributed ledger 110 and include values for the doctor 30 and the patient 20, who are the members (e.g., patient, provider, etc.) involved in this example (Step 13).

Based on the type of intervention, the value of the intervention is assessed (Step 11) for all parties involved. Like all assessment logic, metrics provided to the system 100 are compared to thresholds in the valuation rules 134 to determine the resulting values assigned. For example, the values of therapeutic engagement are scored based upon the candidate's membership in this treatment cohort for a diabetes digital therapeutic. Different conditions may have stricter criteria for valuation and subsequent rewards than others as determined by contracts or payer business criteria as reflected in the valuation rules. At Step 14, the system 100 also orchestrates the process of the payer (40) rewarding the patient (20) and/or provider (30). Selecting a therapeutic reward (46) for the given patient can involve measuring an observation, and optionally any aggregation of past observations, against a threshold goal. In the case of adherence rewards, the “threshold” may be a single observation or may be a number of past observations recorded within a given period of time.

In the counterpart process 200a in the flowchart of FIG. 4B, the distributed network system 100 receives the given therapeutic (medical-related) event for the specified patient 20 (Block 206a), which involves obtaining a diagnosis for the patient 20 from a specific provider 30, thereby incorporating the diagnosis into the distributed ledger 110 (Block 208a).

The disclosed system 100 selects a digital therapeutic program as the chosen intervention in response to the diagnosis based on the intervention rules 132 (Block 210a). The disclosed system 100 then communicates this selected intervention by prescribing the digital therapeutic program to the patient 20 based on the communication rules 122 (Block 212a). Upon receiving consent from the patient 20 to the digital therapeutic program (Block 214a), the disclosed system 100 records this consent and adds an indication of the digital therapeutic program to the distributed ledger 110 (Block 216a).

Subsequently, the disclosed system 100 assesses one or more of the evaluated valuations for both the patient 20 and the provider 30 based on the valuation rules 134 (Block 218a). Finally, the disclosed system 100 adds these assessed valuations for both members 20, 30 to the distributed ledger 110 (Block 220a). Subsequently, the assessed valuation 108 results in the payers (40) performing the settlement (44) with the providers (30) for a value-base contract (50) and also results in the payers (40) providing a reward (46) to the patient (20).

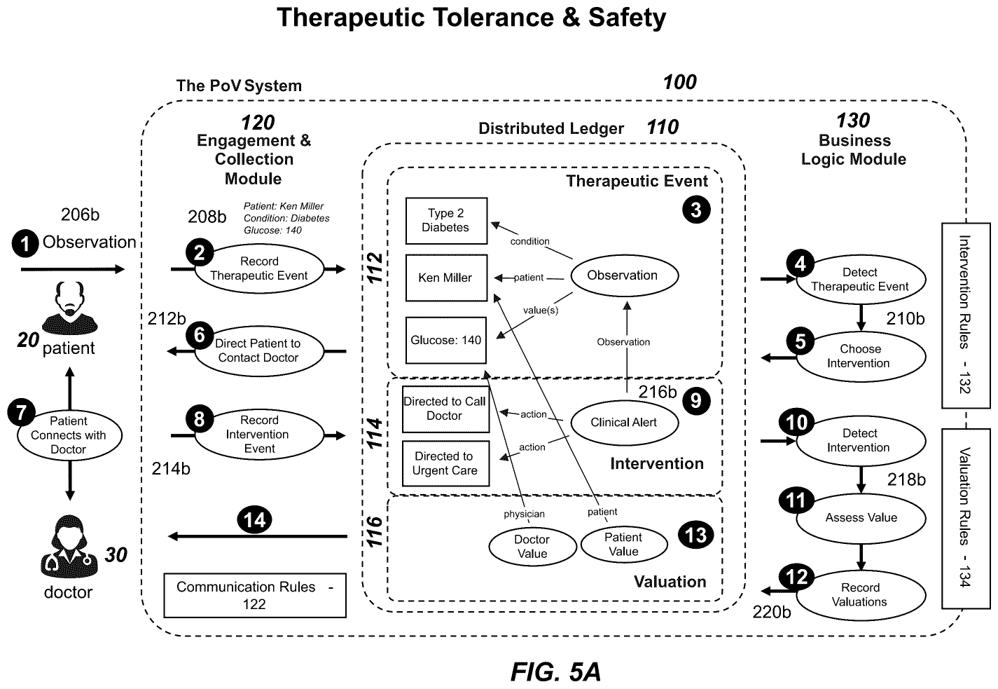

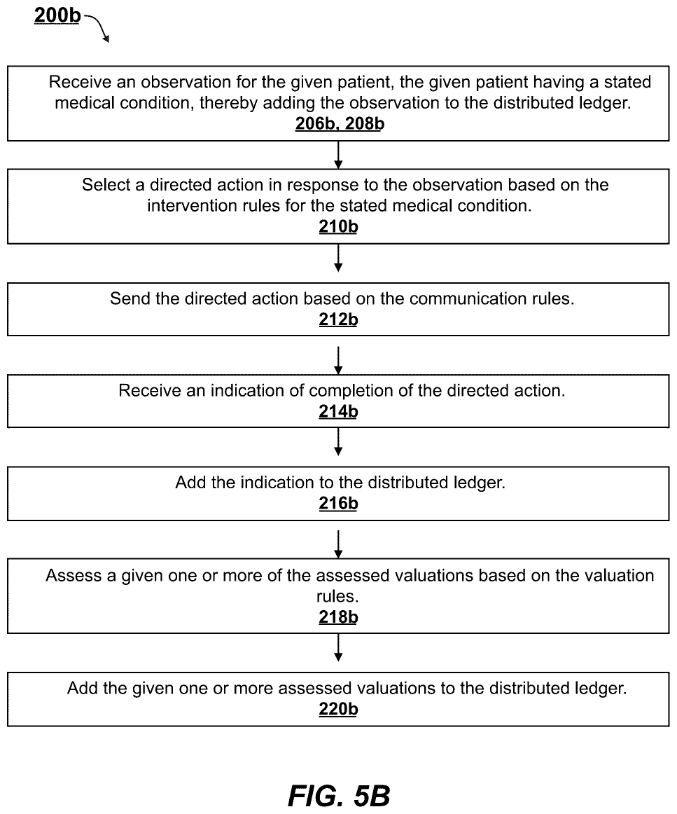

FIG. 5A illustrates an example of the disclosed system 100 being used for therapeutic tolerance and safety. For further understanding, reference is also made to FIG. 5B, which illustrates a portion of the PoV process 200b for the therapeutic tolerance and safety example in FIG. 5A.

During therapeutic tolerance and safety, the patient 20 makes observations using the disclosed system 100 (Step 1). For example, the patient 20 may take glucose readings at home using a glucose monitor, and these glucose readings may be entered into a client device for processing. Alternatively, the glucose monitor may operate automatically as a client device capable of processing according to the disclosed techniques.

Using the communication rules 122, the engagement and collection module 120 records the observations (e.g., medical event) (Step 2) by adding it to the distributed ledger 110 (Step 3). In the provided example, the patient (Ken Miller) has observed a glucose level of 140 mg/dL, which is added to the therapeutic event section 112 of the distributed ledger 110.

The business logic module 130 now detects the therapeutic event (Step 4) and selects intervention(s) for the detected therapeutic event (Step 5). Using the intervention rules 132 governing therapeutics available for the therapeutic event (e.g., observation), a set of interventions may be selected and initiated based upon the type of therapeutic event. In this example, the disclosed system 100 initiates a clinical alert intervention (i.e., a directed action) directing the patient 20 to contact the doctor 30 immediately (Step 6). Like all intervention logic, metrics provided to the system 100 are compared to thresholds in the intervention rules 132 to determine the resulting intervention event. In this case, the patient's glucose level exceeded a safe threshold, requiring the directed action.

Before the selected intervention(s) are added to the distributed ledger 110, the engagement and collection module 120 interacts with the patient 20. In particular, the disclosed system 100 directs the patient 20 to contact his doctor 30 (Step 6). Once the patient 20 connects with the doctor 30 (Step 7), the disclosed system 100 obtains a record, acknowledgement, or other indication of the contact, which can come from the provider 30, and the disclosed system 100 in turn records the intervention event (Step 8) into the intervention section 114 of the distributed ledger 110 (Step 9).

In response to the indication of contact, the business logic module 130 now detects the intervention (Step 10), assesses the value of the intervention (Step 11), and records the valuation(s) into the distributed ledger 110 (Step 12). The valuations are added to the valuation section 116 of the distributed ledger 110 and include physician value and patient value (Step 13). Based on the type of intervention, the value of the intervention is assessed (Step 11) for all parties involved. At Step 14, the system 100 also orchestrates the process of the payer (40) rewarding the patient (20) and/or the provider (30).

Like all assessment logic, metrics are compared to thresholds in the valuation rules 134 to determine the resulting values assigned. For instance, the value of clinical alert engagement can be scored based on the patient's and doctor's performance metric (e.g., time to respond, value of intervention in diminishing the risk of a more serious condition later, etc.). These metrics are assessed against the contract thresholds of the value-based consensus contract 50.

In the counterpart process 200b in the flowchart of FIG. 5B, the distributed network system 100 receives the given therapeutic event for the patient 20, which involves obtaining an observation of the patient 20 who has a specified medical condition (e.g., type 2 diabetes) (Block 206b), thereby incorporating the observation related to the stated medical condition into the distributed ledger 110 (Block 208b).

The distributed network system 100 selects a directed action for the patient 20 in response to the observation based on the intervention rules 132 for the specified medical condition (Block 210b). The disclosed system 100 then communicates this directed action to the patient 20 according to the communication rules 122 (Block 212b). Upon receiving an indication of completion of the directed action (Block 214b), the disclosed system 100 records this completion and adds the indicated completion to the distributed ledger 110 (Block 216b). As will be appreciated, the directed action can include any number of actions to be performed by the patient depending on the stated medical condition and the reported observations.

Subsequently, the distributed network system 100 assesses one or more of the evaluated valuations for both the patient 20 and the provider 30 based on the valuation rules 134 (Block 218b). Finally, the disclosed system 100 adds these assessed valuations for both entities to the distributed ledger 110 (Block 220b).

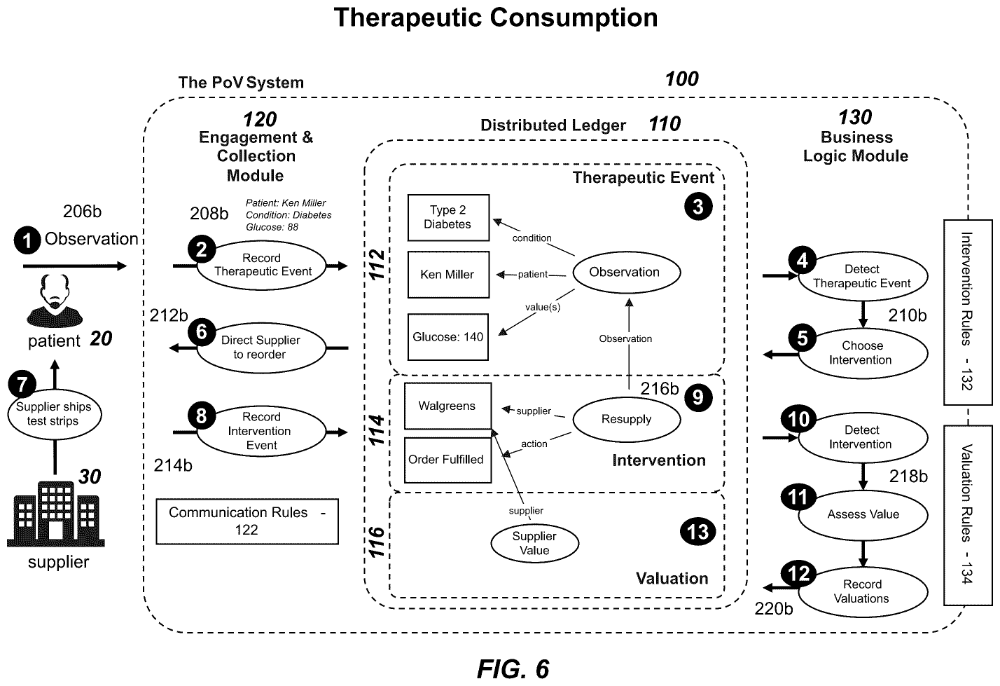

FIG. 6 illustrates an example of the PoV system (e.g., distributed network system) 100 being used for therapeutic consumption. For further understanding, reference can also be made to FIG. 5B, which produces a directed action for the intervention as noted above.

During therapeutic consumption, the patient 20 makes observations using the disclosed system 100 (Step 1). The engagement and collection module 120 records the observations (e.g., medical-related event) (Step 2) by adding it to the distributed ledger 110 (Step 3). In the provided example, the patient (Ken Miller) has observed a test result of a glucose level after a given number of recorded tests. The information of this medical-related event is added to the therapeutic event section 112 of the distributed ledger 110.

The business logic module 130 now detects the medical-related event (Step 4) and selects intervention(s) for the detected event (Step 5). A set of interventions may be selected and initiated based upon the type of medical-related event. In this example, the disclosed system 100 initiates the resupply of test strips with a provider (e.g., supplier) 30 (Step 6). Like all intervention logic, metrics provided to the system 100 are compared to thresholds in the intervention rules 132 to determine the resulting intervention event. In this case, the number of tests is compared against a minimal supply threshold.

Before the selected intervention(s) are added to the distributed ledger 110, the engagement and collection module 120 directs the supplier 30 to reorder test strips for the patient 20. The disclosed system 100 obtains the party's completion of the therapeutic consumption (e.g., when the supplier ships the test slips) (Step 7), and the disclosed system 100 records the intervention event (Step 8) into the intervention section 114 of the distributed ledger 110 (Step 9).

In response to the completion of the intervention event, the business logic module 130 now detects the intervention (Step 10), assesses the value of the intervention (Step 11), and records the valuation(s) into the valuation section 116 of the distributed ledger (Step 12). Based on the type of intervention, the value of the intervention is assessed (Step 11) for all parties involved. In this example, the valuation for the provider (i.e., supplier) 40 (supplier value) is added to the distributed ledger 110. Like all assessment logic, metrics provided to the system are compared to thresholds in the valuations rules 134 to determine the resulting values assigned. For example, the value of resupply is scored based on the supplier's ability to fulfil the order within a time threshold of the contract parameters.

The counterpart process 200b in the flowchart of FIG. 5B can be used for the directed action resulting from therapeutic consumption.

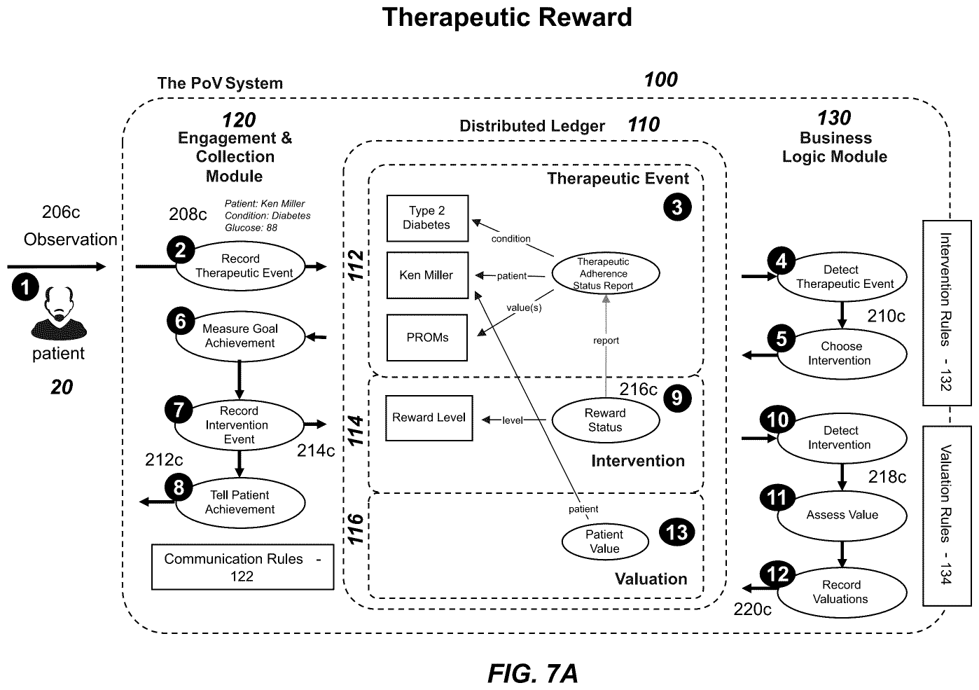

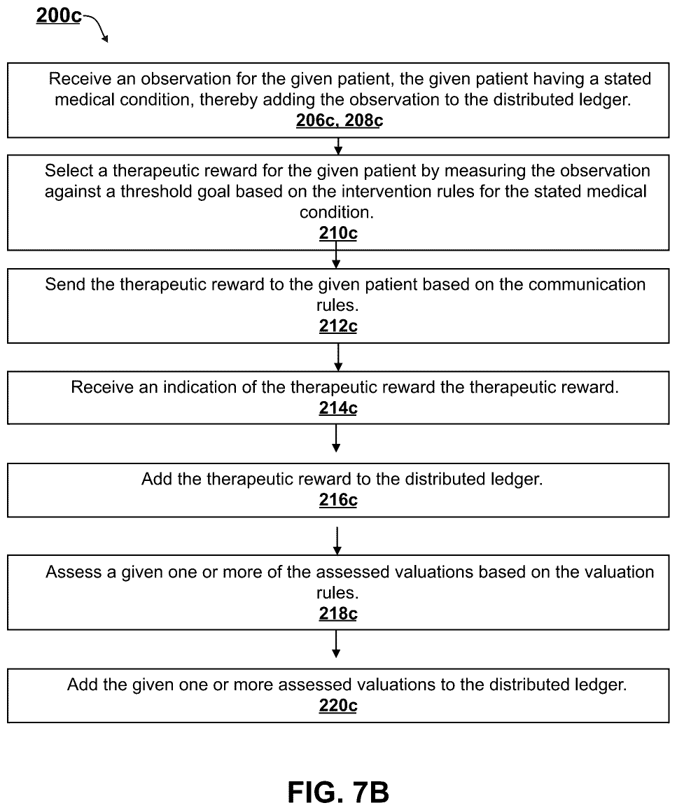

FIG. 7A illustrates an example of the PoV system (e.g., distributed network system) 100 being used for therapeutic reward. For further understanding, reference is also made to FIG. 7B, which illustrates a portion of the PoV process 200c for the therapeutic reward example in FIG. 7A.

During therapeutic reward, the patient 20 makes observations using the disclosed system 100 (Step 1). The engagement and collection module 120 records the observations (e.g., medical-related event) (Step 2) by adding it to the therapeutic event section 112 of the distributed ledger 110 (Step 3). For example, the patient 20 has tested his glucose levels consistently on a scheduled basis according to a therapeutic program for the patient's stated medical condition (type 2 diabetes).

The business logic module 130 now detects the medical-related event (Step 4) and selects intervention(s) for the detected event (Step 5) based on the intervention rules 132. A set of interventions may be selected and initiated based upon the type of medical-related event. In this example, the system 100 determines the patient's adherence to a prescribed therapeutic program to determine the patient's status level in reaching a specific achievement goal for the program, such as completing a consistent set of glucose tests (Step 6). Like all intervention logic, metrics provided to the system 100 are compared to thresholds in the intervention rules 132 to determine the resulting intervention event. In this case, the number or schedule of completed tests are compared against a goal for the program.

The engagement and collection module 120 records the intervention event into the intervention section 114 of the distributed ledger 110 (Step 7) and communicates the achievement to the patient 20 (Step 8).

In response to the completion of the intervention event, the business logic module 130 now detects the intervention (Step 10), assesses the value of the intervention (Step 11) based on the valuation rules 134, and records the valuation(s) into the valuation section 116 of the distributed ledger (Step 12). Based on the type of intervention, the value of the intervention is assessed (Step 11) for all parties involved. In this example, the valuation for the patient 20 (patient value) is added to the distributed ledger 110. Like all assessment logic, metrics provided to the system are compared to thresholds in the valuation rules 134 to determine the resulting values assigned. For example, the reward status for a reward level is added to a Therapeutic Adherence Status Report for the patient 20. The reward can include any number of benefits for the patient 20 based on the value-based consensus contract (50; FIG. 2).

In the counterpart process 200c in the flowchart of FIG. 7B, the distributed network system 100 receives the given medical-related event for the patient 20, which involves obtaining an observation of the patient 20 with a stated medical condition (Block 206c), thus incorporating the observation regarding the stated medical condition into the distributed ledger 110 (Block 208c).

The distributed network system 100 selects a therapeutic reward for the patient 20 as the selected intervention by measuring the observation against a threshold goal, following the intervention rules 132 for the stated medical condition. The disclosed system 100 then communicates this therapeutic reward to the patient 20 per the communication rules 122 (Block 212c). Upon receiving an indication of the therapeutic reward being communicated to the patient 20 (Block 214c), the disclosed system 100 records this indication and adds it as the recorded intervention to the distributed ledger 110 (Block 216c).

Subsequently, the distributed network system 100 assesses one or more of the evaluated valuations for the patient based on the valuation rules 134 (Block 218c). Finally, the disclosed system 100 adds these assessed valuations for the patient 20 to the distributed ledger (Block 220c).

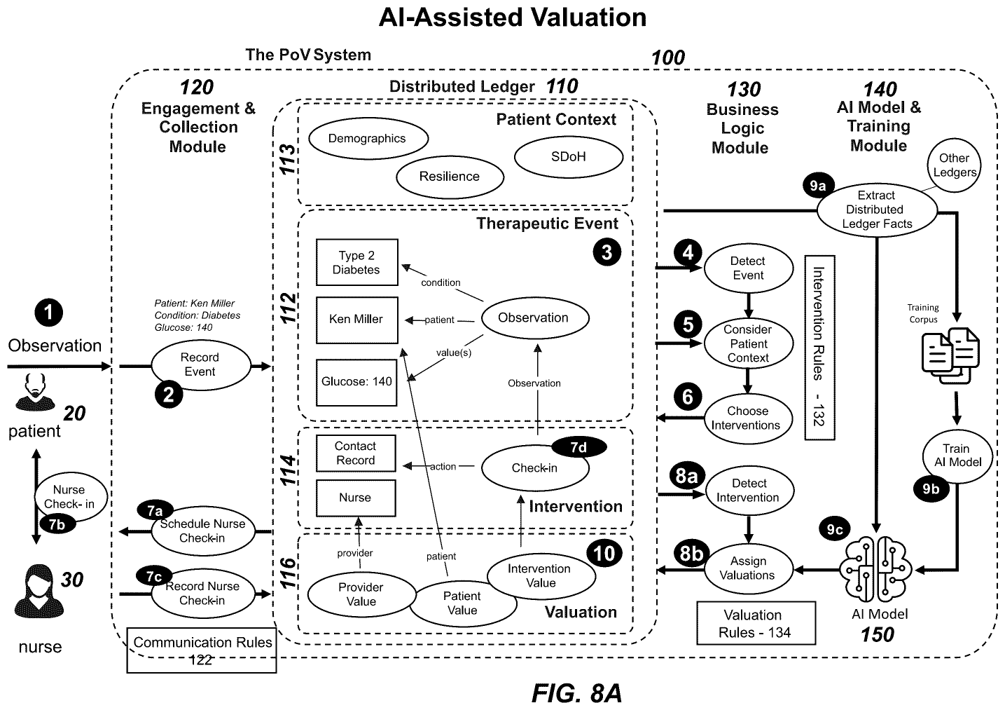

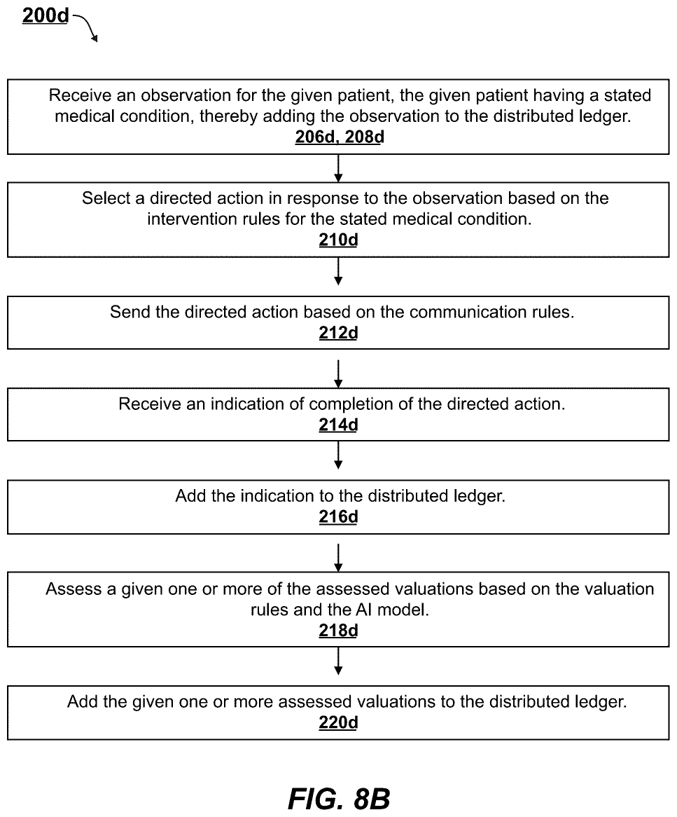

FIG. 8A illustrates an example of the PoV system 100 (e.g., distributed network system) extended to add contextual information about a patient 20 so accurate valuation rules 134 can be applied to intervention participation.

Similar to other configurations, the disclosed PoV system 100 includes a distributed ledger 110 having a therapeutic event section 112, an intervention section 114, and a valuation section 116. The distributed ledger 110 also includes a patient context section 113, which can include extended profile information for the patient (i.e., patient context). In general, the patient context can include the patient's demographic information, resilience of the patient and that of the care network, the community's social determinants of health (SDoH). Other profile information can be included in the patient context, and additional details are provided in the incorporated U.S. Appl. No. ______/______ entitled “System and Method for Therapeutic Event Management and Value Attribution (Proof of Value) in Decentralized Health Information Network.” The PoV system 100 can use the profile information to formulate a strong patient context for the patient 20.

During use, the patient 20 and providers 30 interact with the PoV system 100 in several types of events, including: a medical-related event; a therapeutic-related event; a diagnosis; an observation; a consumption of a medical supply; hospitalization of a care-giver; and a change to a patient's circumstances like financial, housing, or food security; etc. Based on the type of events, the PoV system 100 can in turn provide a set of interventions or actions. To select the best intervention strategy, the PoV system 100 uses available information into the system's algorithms that formulate a course of action.

An example event shown in FIG. 8A includes a therapeutic-related observation made by the patient 20. (As will be appreciated, the PoV system 100 can be used with any number of therapeutic-related events or other types of events.) Initially, the patient 20 makes an observation using the disclosed PoV system 100 (Step 1). The engagement and collection module 120 records the observation (e.g., therapeutic-related event) (Step 2) by adding it to the therapeutic event section 112 of the distributed ledger 110 (Step 3). For example, the patient 20 has tested his glucose level consistently on a scheduled basis according to a therapeutic program for the patient's stated medical condition (type 2 diabetes).

Based on an assessment of the observation, the PoV system 100 prescribes an intervention to occur between the patient 20 and the provider 30 (e.g., nurse). In particular, the business logic module 130 detects observation (the therapeutic-related event) recorded in the therapeutic event section 112 of the distributed ledger 110 (Step 4), and the business logic module 130 considers the patient's context obtained from the patient context section 113 of the distributed ledger 110 (Step 5). In considering the patient's context, the business logic module 130 can consider factual circumstances related to housing, food, and nutrition; transportation; social and economic mobility; education; environmental conditions; concurrent symptoms; past observations and trends; etc. The business logic module 130 then selects one or more interventions based on the detected event and the patient's context according to intervention rules 132 (Step 6).

In general, the PoV system 100 uses the intervention rules 132 governing therapeutics available for the therapeutic-related event (e.g., observation) and selects a set of one or more interventions to be initiated based upon the type of therapeutic-related event and the information in the patent context. Like all intervention logic, metrics provided to the PoV system 100 are compared to thresholds in the intervention rules 132 to determine the resulting interventions. In this example, the patient's glucose level has exceeded a safe threshold, and the PoV system 100 initiates a directed action as an intervention.

As will be appreciated, several directed actions can be initiated. In this example, the directed action for the intervention directs that a scheduled check-in be made between the patient 20 and the provider 30 (e.g., nurse). Before the selected intervention is added to the distributed ledger 110, the engagement and collection module 120 interacts with the nurse 30 by scheduling and communicating a directed action communication to the nurse 30 to check-in with the patient 20 (Step 7A). When the nurse 30 makes that scheduled check-in (Step 7c), the check-in contact is recorded by the PoV system 100 (Step 7c) and is added to the intervention section 114 of the distributed ledger 110 (Step 7d).

With the completion of the intervention, the PoV system 100 then assigns a valuation for the intervention to the valuation section 116 of the distributed ledger 110. Assigning an appropriate valuation can depend on the nature of the intervention involved and all the participants' contributions to the intervention. Because the intervention for the therapeutic-related event is outside a clinical setting, several valuations are possible based on various considerations. For example, one consideration may be based on whether the intervention leads to the best health outcomes for the patient's condition. Another consideration may be based on how well (e.g., how quickly/slowly, how thoroughly, etc.) the participants in the intervention have performed their respective tasks for the intervention.

The assignment of the valuations proceeds as follows in this example. As noted, a clinical alert from the high blood glucose levels from the patient's observations has resulted in the nurse 30 being directed to check-in with the patient 20. The intervention for this directed action may have been made based on trending glucose readings from the patient 20 exceeding a threshold over multiple days. Once the check-in contact has been made, the contact record and identity of the provider are recorded (Step 7d). The disclosed system 100 obtains a record, acknowledgement, or other indication of the contact, which can come from the provider 30, and the PoV system 100 in turn records the intervention event into the intervention section 114 of the distributed ledger 110 (Step 7d).

In response to the indication, the business logic module 130 detects the intervention (Step 8a) and assesses the value of the intervention in the consensus contract (50) based on the valuation rules 134 (Step 8b). As noted above, assessing the valuations in response to the detected intervention can be based on several factors, including the details of the detected therapeutic-related event at Step 4, the patient context at Step 5, and the selected intervention at Step 6, the time frame between actions, and the like. These factors are brought into the algorithm of the valuation rules 134 that formulates the assignment of the valuations (Step 8b). These valuations form the basis and evidence that attributes value in the reconciliation between the provider 30 (revenue cycle management) and the payer (40) (payment integrity processes). The valuations are added to the valuation section 116 of the distributed ledger 110 and can include intervention value, provider value, and patient value, for example (Step 10).

The selection and assignment of valuations can be further enhanced (replaced, supplemented, adjusted, scored, etc.) by an Artificial Intelligence (AI)-assisted valuation model 150, which can be part of an AI module 140 of the disclosed PoV system 100. As shown here, the AI model 150 (e.g., Bayesian network, etc.) can extract facts and other information from the distributed ledger 110 for the patient 20 (Step 9a) and can feed this information into a trained AI model 150 (Step 9c) to select the valuation(s).

The AI model 150 of the AI module 140 can be trained in a training procedure (Step 9b). As briefly shown here for the training, the AI module 140 can extract facts (i.e., factual circumstances, indicators, and the like) from the distributed ledger 110 of this patient and from other sources, such as from the ledgers of other patients. This data forms a training corpus to train the AI model 150 in a training procedure. The AI model 150 is then trained with population level actions, interventions, and future outcomes extracted from distributed ledger facts. The training detects underlying factors within varying patient contexts, conditions, therapeutic programs, and professional care.

Once trained, the trained AI model 150 can be configured to compare performance metrics for interventions against specific factors that result in improved patient outcomes. The AI model 150 can then score different interventions and participant performance metrics based upon the likelihood of improved health outcomes and cost, thus determining valuations for specific interventions to be assigned (Step 8b).

To assign a valuation for the intervention in the present example, the AI model 150 can contain all the patterns of historical intervention paths and participant performance. The details of the completed intervention from the detection (Step 8a) can be valued higher or lower compared to other approaches based on the processing by the AI model 150. Additionally, the valuations recorded in the distributed ledger 110 at Step 10 can include the AI model's rationale with a supporting report of findings.

In the counterpart process 200d in the flowchart of FIG. 8B, the distributed network system 100 receives the given therapeutic-related event for the patient 20, which involves obtaining an observation of the patient 20 with a stated medical condition (Block 206d), thus incorporating the observation regarding the stated medical condition into the distributed ledger 110 (Block 208d).

The distributed network system 100 selects an intervention by measuring the observation against a threshold goal, following the intervention rules 132 for the stated medical condition. The disclosed system 100 then communicates this intervention to the provider 30 (e.g., nurse) per the communication rules 122 (Block 212d). Upon receiving an indication of the recorded intervention being communicated to the provider 30 (Block 214d), the disclosed system 100 records this indication and adds it as the recorded intervention to the distributed ledger 110 (Block 216d).

Subsequently, the distributed network system 100 assesses one or more of the evaluated valuations for the patient based on the valuation rules 134 and based on the AI model 150 (Block 218d). For example, the AI model 150 can replace, structure, score, or supplement the valuation rules 134. Finally, the disclosed system 100 adds these assessed valuations for the provider 30 to the distributed ledger 110 (Block 220d).

Several types of AI models 150 can be used for the techniques of the present disclosure, including, for example, a symbolic AI model, a Bayesian network model, a machine learning model, a neural network model, and types of algorithmic models. A few of these models are briefly described.

As is known, a Bayesian network (a.k.a. a belief network or a causal probabilistic network) is a graphical model representing probabilistic relationships among a set of variables. The model includes nodes (representing variables) and includes directed edges (representing probabilistic dependencies) between the variables. At its core, the Bayesian network provides a joint probability distribution of the variables in a compact form by employing conditional independence relationships.

Each node in the Bayesian network represents a random variable and is associated with a conditional probability distribution that quantifies the probability of that variable given its parent variables. The parent variables of a node are the variables that directly influence the node's probability distribution. By specifying these conditional probabilities for each node, the Bayesian network encodes the entire joint probability distribution of the variables.

For their part, the directed edges in the Bayesian network define causal relationships or dependencies among variables. The direction of the edges indicates the direction of influence: from parent variables to child variables. This structure enables efficient inference by allowing reasoning about the probabilities of variables given observed evidence or interventions on certain variables.

A symbolic AI model uses explicit rules and logical reasoning to solve problems. In the symbolic AI model, knowledge is represented in a structured format, such as rules or ontologies, and algorithms manipulate these rules to derive conclusions or make decisions. The knowledge can be encoded as rules or if-then statements to provide recommendations or solutions.

A machine learning model learns from data to improve performance over time without being explicitly programmed. Several subtypes, including supervised learning, unsupervised learning, and reinforcement learning, can be used. Supervised learning involves training a model using labeled data, where the input-output relationships are explicitly provided. Supervised learning is suitable for classification and regression types of tasks. Unsupervised learning trains models using unlabeled data to discover hidden patterns or structures within the data and is suitable for clustering and dimensionality reduction. In reinforcement learning, an agent learns to make sequential decisions by interacting with an environment and receiving feedback in the form of rewards or penalties.

A neural network model has interconnected nodes, or neurons, organized into layers. Each neuron applies a mathematical operation to its inputs and passes the result to the next layer. Deep learning as a subset of a neural network trains models with multiple layers in a deep architecture to learn hierarchical representations of the data. Convolutional neural networks (CNNs) are suitable for image recognition and computer vision, whereas recurrent neural networks (RNNs) are suited to analyzing sequential tasks, such as natural language processing and time series prediction.

An algorithmic model iteratively evolves candidate solutions using mechanisms such as mutation, crossover, and selection. The algorithmic model is useful for problems having large search spaces or complex, multi-dimensional objective functions.

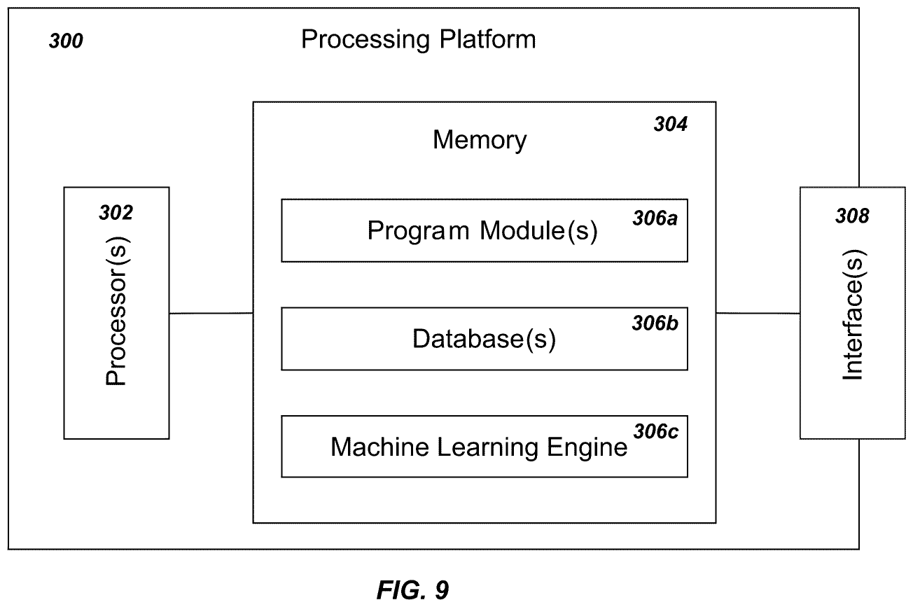

As one example according to the present disclosure, FIG. 9 illustrates a processing platform 300 for preparing training data to be used in training the AI model (150 of FIG. 8A) of the present disclosure in a computing environment. As noted, the AI model (150) can be a Bayesian network. The processing platform 300 can maintain a model that the processing platform 300 may use to prepare the training data. Moreover, the processing platform 300 can dynamically update the training data as additional inputs, rules, restrictions, etc. are received. Additionally, because medical information is used in the PoV system (100) of the present disclosure, the processing platform 300 can use large language models (LLMs) to understand, process, and generate language and perform tasks that construct, arrange, and manage medical information to be used as training data for the AI model (150).

In general, the processing platform 300 can be associated with an organization or entity (e.g., a health care company, a research entity, an insurance agency, or the like). The processing platform 300 may use one or more computing devices configured to perform one or more of the functions described herein. For example, the processing platform 300 may use one or more computers (e.g., servers, server blades, laptop computers, desktop computers, etc.). As generally shown here, the processing platform 300 can include one or more processors 302, memory 304, and an interface 306. A data bus can interconnect the processor 302, the memory 304, and the interface 306.

With respect to the one or more processors 302, the processing platform 300 can be powered by processing units capable of handling the computational demands of Bayesian inference. These processing units can be optimized for machine learning tasks to accelerate probabilistic computations.

With respect to the memory 304, the memory 304 may require sufficient Random Access Memory (RAM) for storing the various states and data required during Bayesian inference and training. The memory allocation should have sufficient resources available for real-time inference and learning processes to be performed by the processing platform 300. The memory 304 may include one or more program modules 306a having instructions that when executed by the one or more processors 302 cause the processing platform 300 to perform one or more functions. The memory 304 may also include one or more databases 306b that may store and/or otherwise maintain information which the program modules 306a may use.

In some instances, the one or more program modules 306a and/or databases 306b may be stored by and/or maintained in different memory units of the processing platform 300 and/or by different computing devices that may form and/or otherwise make up the processing platform 300. For the one or more databases 306b, for example, the processing platform 300 can integrate with both structured and unstructured data storage systems, which may include SQL-based relational databases (e.g., PostgreSQL, MySQL) for structured data and NoSQL databases (e.g., MongoDB, Cassandra) for unstructured data. Data sources (not shown) are linked to the Bayesian network of the AI model (150) so vast amounts of information can be processed for both training and real-time decision-making.

As noted above, the processing platform 300 can be configured to perform natural language processing techniques to prepare training data of medical information to be used to train the AI model (150). Accordingly, the processing platform 300 may have instructions that direct and/or cause the processing platform 300 to execute advanced natural language processing techniques. In particular, the processing platform 300 can be configured to perform natural language processing techniques to handle medical facts for the training data used in training the AI model (150) according to the present disclosure. The processing platform 300 can apply such natural language processing techniques to de-identify identifiable facts and to alter (fictionalize) other facts. Therefore, the memory 304 may include one or more modules 306a and databases 306b used for natural language processing.

In general, the machine learning engine 306c is configured to facilitate the training and inference of AI model (150) (e.g., the Bayesian network). The machine learning engine 306c performs the probabilistic computations, parameter learning, and structure learning to optimize the network of the AI model (150) to the trained AI model (150) can more accurately reflect underlying probability distributions based on input data. The machine learning engine 306c can use open-source frameworks, such as TensorFlow Probability, PyTorch, or specialized Bayesian network software (e.g., Hugin or GeNIe).

Finally, the one or more interfaces 308 can include a network interface configured to support communication between the processing platform 300 and one or more networks (not shown). In general, the one or more interfaces 308 can include both graphical and programmatic interfaces for user interaction and automation. The user interface (UI) can allow users to visualize the Bayesian network, input data, and interpret outputs, often through dashboards or specialized software interfaces. Additionally, the one or more interfaces 308 can include Application Programming Interfaces (API) so the processing platform 300 can integrate with external systems, enabling automated data ingestion, inference requests, and output extraction for downstream applications.

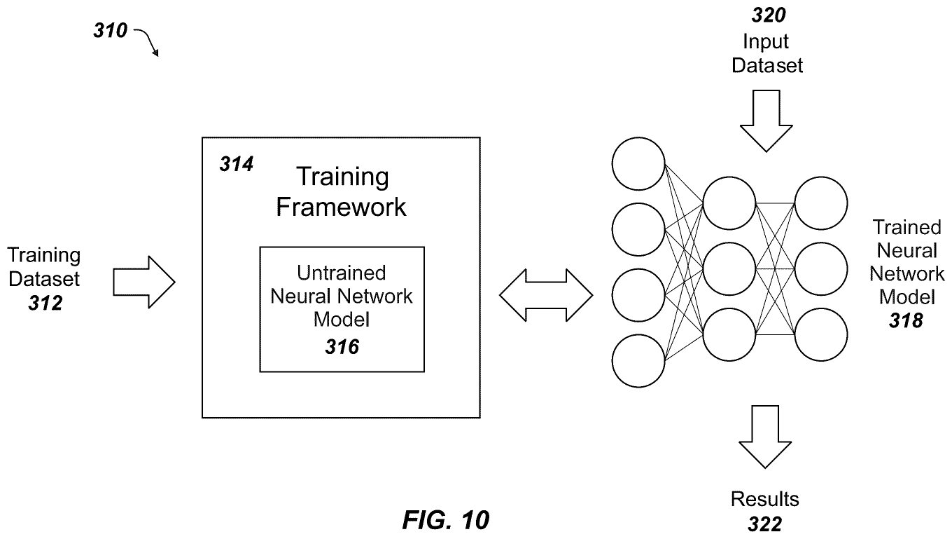

As a further example according to the present disclosure, FIG. 10 illustrates a process for training and deploying a trained neural network model 318 of a deep neural network 310. The following section generally describes the training of an untrained neural network model 316 to generate the trained neural network model 318 using a training dataset 312. The training can be used to train an AI model (150) of the disclosed PoV system (100) used in formulating interventions based on a patient's context (factual circumstances) as described above.

Once the neural network 310 has been structured for a task, the neural network 310 is trained using a dataset of training dataset 312. Because medical facts may be present, the training dataset 312 of medical information can be initially processed to remove any personally identifiable facts from the information.

Once the training dataset 312 is available, training of the neural network 310 can begin. To begin the training, initial weights of an untrained model 316 may be chosen randomly or by pre-training using a deep belief network. The training framework 314 then performs a training cycle, which can then be performed in either a supervised or unsupervised manner.

Supervised learning uses the training dataset 312 to teach models to yield the desired output. The training dataset 312 includes inputs and desired outputs, which allow the neural network 310 to learn over time. Alternatively, when the training dataset 312 includes input having known output, the output of the neural network 310 can be manually graded.

The training framework 314 of the neural network 310 processes the inputs and compares the resulting outputs against a set of expected or desired outputs. Errors are then propagated back through the training framework 314. As the training proceeds, the training framework 314 can adjust and change the weights that control the untrained model 316. The training framework 314 can provide tools to monitor how well the untrained model 316 is converging towards a trained model suitable for generating correct answers based on known input data. The training process repeats as the network weights are adjusted to refine the output generated by the training framework 314. The training process can continue until the neural network 310 reaches a statistically desired accuracy associated with a trained model 318. Ultimately, the trained model 318 can then be deployed in the disclosed PoV system (100) so the trained model 318 presented with an input dataset 320 can implement the machine learning operations and output a result 322 for use according to the purposes of the disclosed PoV system (100).

Supervised learning is typically separated into two types of problems—classification and regression. Classification uses an algorithm to assign test data accurately into specific categories. Regression is used to understand the relationship between dependent and independent variables. Numerous different algorithms and computation techniques can be used in supervised machine learning, including but not limited to, neural networks, naïve bayes, linear regression, logistic regression, support vector machines (SVM), k-nearest neighbor, and random forest.

Unsupervised learning is a learning method in which the network uses algorithms to analyze and cluster unlabeled data. These algorithms discover hidden patterns or data groupings. Therefore, the training dataset 312 includes input data without any associated output data. The untrained model 316 can learn groupings within the unlabeled input and determine how individual inputs relate to the overall dataset.

Unsupervised training can be used for three main tasks—clustering, association, and dimensionality. Clustering is a data mining technique that groups unlabeled data based on similarities and differences. This technique is often used to process raw, unclassified data objects into groups represented by structures or patterns in the information. Association is a rule-based method for finding relationships between variables in a given dataset. This method is often used for market basket analysis. Dimensionality reduction is used when a given dataset's number of features (dimensions) is too high. This technique is commonly used in the preprocessing of data.

Variations of supervised and unsupervised training may also be employed. Semi-supervised learning is a technique in which the training dataset 312 includes a mix of labeled and unlabeled data of the same distribution. Incremental learning is a variant of supervised learning in which input data is continuously used to train the model further. Incremental learning enables the trained model 318 to adapt to the input dataset 320 without forgetting the knowledge instilled within the neural network 310 during initial training.

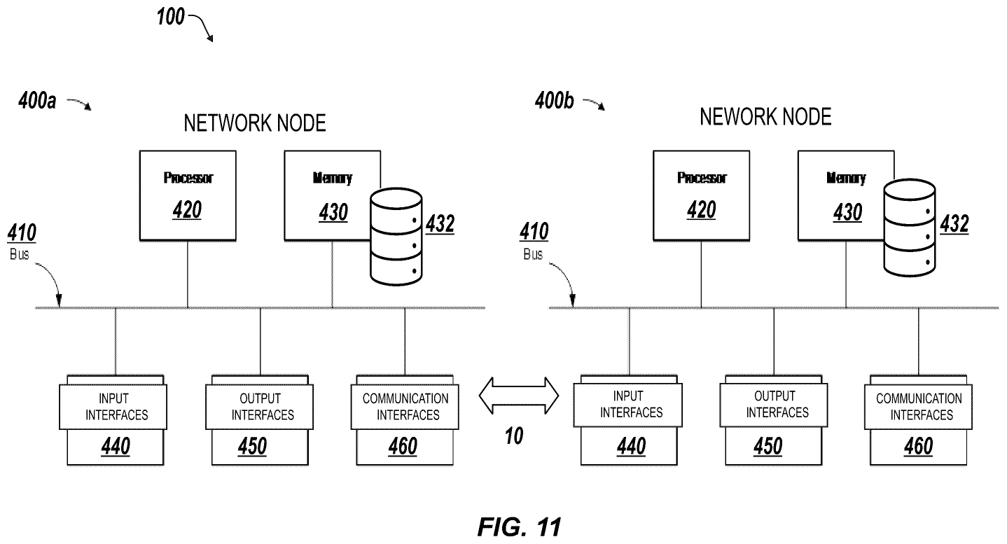

FIG. 11 schematically illustrates components of network devices or nodes 400a-b of the disclosed system 100 in the network system 10 of FIGS. 1-2. As shown in FIG. 11, each network node 400a-b may include a bus 410, a processor 420, a memory 430, and a communication interface 440. Other components, such as an input and output interface 440, 450, may be available.

The bus 410 includes one or more components that enable wired and/or wireless communication among the components of the network node 400a-b. The bus 410 may couple together two or more components of FIG. 11, such as via operative coupling, communicative coupling, electronic coupling, and/or electric coupling. The processor 420 includes a central processing unit, a microprocessor, a controller, a microcontroller, a digital signal processor, a field-programmable gate array, an application-specific integrated circuit, and/or another type of processing component. The processor 420 is implemented in hardware, firmware, or a combination of hardware and software. In some implementations, the processor 420 includes one or more processors capable of being programmed to perform one or more operations or processes described elsewhere herein.

The memory 430 includes volatile and/or nonvolatile memory. For example, the memory 430 may include random access memory (RAM), read only memory (ROM), a hard disk drive, and/or another type of memory (e.g., a flash memory, a magnetic memory, and/or an optical memory). The memory 430 may include internal memory (e.g., RAM, ROM, or a hard disk drive) and/or removable memory (e.g., removable via a universal serial bus connection). The memory 430 may be a non-transitory computer-readable medium. The memory 430 stores information, instructions, and/or software (e.g., one or more software applications) related to the operation of the network node 400a-b. In some implementations, the memory 430 includes one or more memories that are coupled to one or more processors (e.g., the processor 420), such as via the bus 410.

For at least one network node 400a, the input/output interfaces 440, 450 enables the network node 400a to receive input, such as user input and/or sensed input. For example, the input/output interfaces 440, 450 may include a touch screen, a keyboard, a keypad, a mouse, a button, a microphone, a switch, a sensor, etc. The input/output interfaces 440, 450 also enables the network node 400a to provide output, such as via a display, a speaker, etc.

The communication interface 440 enables the network node 400a-b to communicate with other devices via a wired connection and/or a wireless connection. For example, the communication interface 440 may include a receiver, a transmitter, a transceiver, a modem, a network interface card, and/or an antenna.

The network node 400a-b may perform one or more operations or processes described herein. For example, a non-transitory computer-readable medium (e.g., the memory 430) may store a set of instructions (e.g., one or more instructions or code) for execution by the processor 420. The processor 420 may execute the set of instructions to perform one or more operations or processes described herein. In some implementations, execution of the set of instructions, by one or more processors 420, causes the one or more processors 420 and/or the network node 400a-b to perform one or more operations or processes described herein. In some implementations, hardwired circuitry may be used instead of or in combination with the instructions to perform one or more operations or processes described herein. Additionally, or alternatively, the processor 420 may be configured to perform one or more operations or processes described herein. Thus, implementations described herein are not limited to any specific combination of hardware circuitry and software.

As disclosed herein, the PoV system (e.g., distributed network system) 100 operates within one or more networks of a decentralized health information network system 10, connecting with numerous network nodes, which are associated with patients, providers, and payers, to facilitate PoV transaction fulfillment based on a consensus contract. The several components for the disclosed system 100 can be incorporated into one or more of the network nodes 400a-b, can be shared between one or more of the network nodes 400a-b, and/or can be distributed among several of the network node 400a-b. (Although two network node 400a-b are shown, it will be appreciated that the network can include any plurality of such devices.) Therefore, the disclosed system 100 generally includes one or more databases 332 (e.g., in memory 430), one or more interfaces (e.g., 440, 450, 460), and one or more processors (e.g., processors 420) incorporated into one or more of the network node 400a-b, shared between several of the network node 400a-b, and/or distributed among several of the network node 400a-b.

The one or more databases 332 in memory 430 store intervention rules, valuation rules, and communication rules, all governed by the consensus contract (50; FIG. 2). These rules dictate interventions in response to medical-related events, attribute valuations to network nodes (11), and define communication protocols with each network node (11). The one or more interfaces 440, 450, 460 are designed to facilitate communication between network nodes (11) across the networks (12).

The one or more processors 420 are in operational communication with the databases 332 and the interfaces 440, 450, 460. These processors 420 are configured to build a distributed ledger (110) for a given patient (20). The distributed ledger (110) is a trusted repository of information because all parties have a copy of the distributed ledger (110), and a consensus protocol dictates how to add or modify ledger entries in the distributed ledger (110), ensuring its integrity. To build the distributed ledger (110), the processors 420 receive medical-related or other types of events for the given patient (20), add these events to the distributed ledger 110 based on communication rules; select interventions based on intervention rules; communicate these interventions to relevant network nodes (11); record the interventions in response to the communication; and evaluate the interventions' value for the involved members (e.g., patient, provider, etc.) according to the valuation rules. Subsequently, the assessed valuations are added to the distributed ledger (110).

Furthermore, the processors 420 are configured to distribute the distributed ledger (110) within the health information network system 10. Additionally, the processors 420 are configured to arrange settlements (44) or rewards (46) for the assessed valuations among the relevant members (e.g., patient, provider, etc.) in accordance with the consensus contract (50). Overall, the disclosed system 100 actively performs these functions to ensure seamless operation and adherence to the established consensus contract (50).

The number and arrangement of components shown in FIG. 11 are provided as an example. The network node 400a-b may include additional components, fewer components, different components, or differently arranged components than those shown in FIGS. 11. Additionally, or alternatively, a set of components (e.g., one or more components) of the network node 400a-b may perform one or more functions described as being performed by another set of components of the network node 400a-b.

The techniques of the present disclosure can be implemented in digital electronic circuitry, or in computer hardware, firmware, software, or in combinations of these. Apparatus for practicing the disclosed techniques can be implemented in a computer program product tangibly embodied in a machine-readable storage device for execution by a programmable processor; and method steps of the disclosed techniques can be performed by a programmable processor executing a program of instructions to perform functions of the disclosed techniques by operating on input data and generating output. The disclosed techniques can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random-access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

The foregoing description of preferred and other embodiments is not intended to limit or restrict the scope or applicability of the inventive concepts conceived of by the Applicants. It will be appreciated with the benefit of the present disclosure that features described above in accordance with any configuration or aspect of the disclosed subject matter can be utilized, either alone or in combination, with any other described feature, in any other configuration or aspect of the disclosed subject matter.

In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

1. A method, used in a distributed network system between a plurality of network nodes in a healthcare environment, to fulfill proof of value transactions according to a consensus contract, the network nodes being at least associated with one or more members in the healthcare environment that include patients, providers, and payers, the method comprising
storing, in one or more databases of the distributed network system, (i) a plurality of intervention rules governed by the consensus contract, the intervention rules defining a plurality of interventions in response to events, (ii) a plurality of valuation rules governed by the consensus contract, the valuation rules attributing a valuation for each of the members for the interventions, and (iii) a plurality of communication rules governed by the consensus contract, the communication rules defining a plurality of communications with each of the network nodes for the interventions;
building, with one or more processors of the distributed network system, a distributed ledger for a given patient by:
receiving a given event for the given patient;
adding the given event to the distributed ledger based on the communication rules;
selecting a given intervention, as a selected intervention, in response to the given event based on the intervention rules;
communicating the selected intervention, as a communicated intervention, to a given one of the network nodes based on the communication rules;
receiving a record of the communicated intervention, as a recorded intervention, in response to the communication;
adding the recorded intervention to the distributed ledger in response to the record;
assessing one or more given valuations, as one or more assessed valuations, of the recorded intervention for a given one or more of the members based on the valuation rules; and
adding the one or more assessed valuations to the distributed ledger; and
configuring a settlement of the one or more assessed valuations for the given one or more of the members according to the consensus contract by distributing the distributed ledger in the distributed network system.

2. The method of claim 1, wherein the intervention rules defining the interventions in response to the events define how each of the events are detected and how the interventions are subsequently selected.

3. The method of claim 1, wherein the valuation rules attributing the valuation for each of the given one or more members for the interventions define how the recorded interventions are detected and how the recorded interventions are subsequently valued.

4. The method of claim 1, wherein the communication rules defining the communications with each of the network nodes for the interventions define how the distributed network system interfaces with each of the network nodes in sending and receiving the communications.

5. The method of claim 1, wherein receiving the given event for the given patient comprises receiving a diagnosis for the given patient from a given provider, thereby adding the diagnosis to the distributed ledger.

6. The method of claim 5, wherein:
selecting comprises selecting a digital therapeutic program, as the selected intervention, in response to the diagnosis based on the intervention rules;
communicating comprises communicating the digital therapeutic program, as the communicated intervention, to the given patient based on the communication rules;
receiving comprise receiving a consent from the given patient to the digital therapeutic program, as the record, in response to the communication; and
adding comprises adding an indication of the digital therapeutic program, as the recorded intervention, to the distributed ledger in response to the consent.

7. The method of claim 6, wherein:
assessing comprises assessing a given one or more of the assessed valuations for both the given patient and the given provider based on the valuation rules; and
adding comprises adding the given one or more assessed valuations for both the given patient and the given provider to the distributed ledger.

8. The method of claim 1, wherein receiving the given event for the given patient comprises receiving an observation of the given patient, the given patient having a stated medical condition, thereby adding the observation for the stated medical condition to the distributed ledger.

9. The method of claim 8, wherein:
selecting comprises selecting a directed action for the given patient to contact a given provider, as the selected intervention, in response to the observation based on the intervention rules for the stated medical condition;
communicating comprises communicating the directed action, as the communicated intervention, to the given patient based on the communication rules;
receiving comprise receiving an indication of completion of the directed action, as the record, in response to the communication; and
adding comprises adding the indication of completion of the directed action, as the recorded intervention, to the distributed ledger in response to indication.

10. The method of claim 9, wherein:
assessing comprises assessing a given one or more of the assessed valuations for the given patient and the given provider based on the valuation rules; and
adding comprises adding the given one or more assessed valuations for both the given patient and the given provider to the distributed ledger.

11. The method of claim 8, wherein:
selecting comprises selecting a directed action for a given provider, as the selected intervention, in response to the observation based on the intervention rules for the stated medical condition;
communicating comprises communicating the directed action, as the communicated intervention, to the given provider based on the communication rules;
receiving comprise receiving an indication of completion of the directed action, as the record, in response to the communication; and
adding comprises adding the indication of completion of the directed action, as the recorded intervention, to the distributed ledger in response to the indication.

12. The method of claim 11, wherein:
assessing comprises assessing a given one or more of the assessed valuations for the given provider based on the valuation rules; and
adding comprises adding the given one or more assessed valuations for the given provider to the distributed ledger.

13. The method of claim 8, wherein:
selecting comprises selecting a therapeutic reward for the given patient, as the selected intervention, by measuring the observation, and optionally any past observations, against a threshold goal based on the intervention rules for the stated medical condition;
communicating comprises communicating the therapeutic reward, as the communicated intervention, to the given patient based on the communication rules;
receiving comprise receiving an indication of the therapeutic reward, as the record, in response to the communication; and
adding comprises adding the therapeutic reward, as the recorded intervention, to the distributed ledger in response to the indication.

14. The method of claim 13, wherein:
assessing comprises assessing a given one or more of the assessed valuations for the given patient based on the valuation rules; and
adding comprises adding the given one or more assessed valuations for the given patient to the distributed ledger.

15. The method of claim 1, wherein configuring the settlement by distributing the distributed ledger comprises:
adding data including the given event, the recorded intervention, and the one or more assessed valuations in blocks to the distributed ledger based on a consensus protocol dictating how to add or modify entries in the distributed ledger; and
automatically fulfilling terms of the consensus contract in response to predefined conditions being met by coding terms of the consensus contract into code used in the distributed network system.

16. The method of claim 1, wherein to assess the one or more assessed valuations, the method comprises using an artificial intelligence (AI) model.

17. The method of claim 16, wherein to use the AI model, the method comprises:
training the AI model to score a plurality of interventions based on performance metrics; and
informing the step of assessing the one or more given valuations based on the scoring by the AI model to formulate the one or more assessed valuations for the given one or more of the members.

18. The method of claim 1, wherein the providers include one or more of a hospital, a doctor, a medical supplier, etc.; wherein the payers include one or more of health management company, a medical insurer, an insurance company, a health plan administrator, etc.; wherein the events include one or more of: a medical-related event, a therapeutic-related event, a diagnosis, a medical observation, a consumption of a medical supply, hospitalization of a care-giver, and a change to a patient's circumstances like financial, housing, or food security; and wherein the interventions include one or more of a therapeutic protocol; a directed action, an adjustment to care plan, an educational material to raise condition literacy, an assistance to raise awareness about social program (like ride assistance or meal delivery), a delivery of a medical supply, and a reward for achieving therapeutic goals (like points programs, or reduced insurance cost).

19. A programmable storage device having program instructions stored thereon for causing one or more programmable network devices to perform a method of claim 1, used in a distributed network system between a plurality of network nodes, to fulfill transactions according to a consensus contract.

20. A distributed network system used in one or more networks between a plurality of network nodes to fulfill proof of value transactions according to a consensus contract, the network nodes being at least associated with members in a healthcare environment that includes patients, providers, and payers, the distributed network system comprising:
one or more databases storing (i) a plurality of intervention rules governed by the consensus contract, the intervention rules defining a plurality of interventions in response to events, (ii) a plurality of valuation rules governed by the consensus contract, the valuation rules attributing a valuation for each of the members for the interventions, and (iii) a plurality of communication rules governed by the consensus contract, the communication rules defining a plurality of communications with each of the network nodes for the interventions;
one or more interfaces configured to communicate the network nodes with one another via the one or more networks; and
one or more processors in operable communication with the one or more databases and the one or more interfaces,
wherein, to build a distributed ledger for a given patient, the one or more processors are configured to:
receive a given event for the given patient;
add the given event to the distributed ledger based on the communication rules;
choose a given intervention, as a selected intervention, in response to the given event based on the intervention rules;
communicate the selected intervention, as a communicated intervention, to a given one of the network nodes based on the communication rules;
receive a record of the communicated intervention, as a recorded intervention, in response to the communication;
add the recorded intervention to the distributed ledger in response to the record;
assess one or more given valuations, as one or more assessed valuations, of the recorded intervention for a given one or more of the members based on the valuation rules; and
adding the one or more assessed valuations to the distributed ledger; and
wherein the one or more processors are configured to:
distribute the distributed ledger in the distributed network system; and
configure a settlement of the one or more assessed valuations for the given one or more of the members according to the consensus contract.
